(19) United States
(12) Patent Application Publication
(10) Pub. No.: US 2024/0240248 A1
(43) Pub. Date: Jul. 18, 2024

(54) METHODS FOR COMPLEMENT STRAND SEQUENCING

(71) Applicant: Oxford Nanopore Technologies PLC, Oxford (GB)

(72) Inventors: Paul Richard Moody, Oxford (GB); Jason Robert Hyde, Oxford (GB); James Anthony Clarke, Oxford (GB)

(73) Assignee: Oxford Nanopore Technologies PLC, Oxford (GB)

(21) Appl. No.: 18/561,834

(22) PCT Filed: May 19, 2022
(86) PCT No.: PCT/GB2022/051269

§ 371 Date: Nov. 17, 2023

Related U.S. Application Data
(60) Provisional application No. 63/190,689, filed on May 19, 2021.

(51) Int. Cl. C12Q 1/6869

(52) U.S. Cl. CPC C12Q 1/6869

(57) ABSTRACT

Aspects of the disclosure relate to compositions and methods for characterizing nucleic acids using a nanopore. The disclosure is based, in part, on methods for increasing follow-on sequencing of nucleic acid strands. In some embodiments, the methods comprise increasing the concentration of a tethering agent. In some embodiments, the methods comprise use of adaptors having a rigid (or stiffened) leader section. Compositions and systems including, e.g., adaptors for attachment to double-stranded poly nucleotides and/or tethering agents, which can be used in the methods are also provided.

### RELATED APPLICATIONS

This application is a national stage filing under 35 U.S.C. § 371 of international PCT application PCT/GB2022/051269, filed May 19, 2022, which claims priority under 35 U.S.C. § 119(e) to U.S. provisional patent application, U.S. Ser. No. 63/190,689, filed May 19, 2021, the entire contents of each of which are incorporated by reference herein.

### REFERENCE TO AN ELECTRONIC SEQUENCE LISTING

The contents of the electronic sequence listing (0036670116US01-SEQ-KZM.txt; Size: 3,606 bytes; and Date of Creation: Nov. 17, 2023) is herein incorporated by reference in its entirety.

### BACKGROUND

There is currently a need for rapid and cheap polynucleotide (e.g., DNA or RNA) sequencing and identification technologies across a wide range of applications. Strand sequencing typically involves the use of a polynucleotide binding protein such as a helicase to control the movement of the polynucleotide through the nanopore. Double stranded polynucleotides may be determined by separating the strands to provide single stranded polynucleotides prior to translocation through the nanopore. The two strands of the double stranded polynucleotide may be linked by a bridging moiety such as a hairpin loop in order to ensure that translocation of the forward (template) strand is followed by translocation of the reverse (complement) strand. However, preparation of such a hairpin-linked polynucleotide can increase sample preparation time and result in a loss of valuable analyte. Further, translocation of a hairpin linked template and complement polynucleotide strands through a nanopore can give rise to rehybridization of the strands on the other (trans) side of the nanopore. This can alter the rate of translocation giving rise to a lower sequencing accuracy.

### SUMMARY

Aspects of the disclosure relate to compositions and methods for characterizing nucleic acids using a nanopore. The disclosure is based, in part, on methods for increasing follow-on sequencing of nucleic acid strands. Compositions and systems including, e.g., adaptors for attachment to double-stranded polynucleotides and/or tethering agents, which can be used in the methods are also provided.

In some aspects, the disclosure provides a method comprising adding a plurality of tethers to a well comprising a nanopore disposed in a membrane wherein the concentration of tethers added to the well is at least 100 nM; contacting the nanopore with a double stranded nucleic acid complex comprising a pair of non-covalently bound single stranded nucleic acids, each single stranded nucleic acid of the pair comprising an adaptor having a leader; and applying a potential to the membrane to promote translocation of the single stranded nucleic acids through the nanopore.

In some embodiments, the first nucleic acid and the second nucleic acid of the pair are each DNA or RNA. In some embodiments, the first nucleic acid and second nucleic acid of the pair are complementary to one another.

In some embodiments, the adaptor of a first single stranded nucleic acid of the pair is positioned on the 5′ end of the first single stranded nucleic acid. In some embodiments, the adaptor of a second single stranded nucleic acid of the pair is positioned on the 5′ end of the second single stranded nucleic acid.

In some embodiments, each leader comprises one or more poly-dT section. In some embodiments, each leader comprises two or more poly-dT sections, optionally wherein each of the poly-dT sections are non-contiguous.

In some embodiments, each adaptor further comprises one or more spacers. In some embodiments, each of the one or more spacers are selected from an iSp3C spacer, iSpC9 spacer, and iSpC18 spacer.

In some embodiments, each adaptor further comprises one or more modified nucleotides. In some embodiments, the modified nucleotides are 2′-O-Methyl (2′OMe) modified nucleotides.

In some embodiments, the nanopore is a protein nanopore, optionally wherein the nanopore is a CsgG nanopore.

In some embodiments, each of the tethers is a lipid, fatty acid, sterol, carbon nanotube, polypeptide, protein or amino acid. In some embodiments, each of the tethers comprises a lipid, fatty acid, sterol, carbon nanotube, polypeptide, protein or amino acid. In some embodiments, each of the tethers comprises tocopherol. In some embodiments, each of the tethers comprises octyl-tocopherol. In some embodiment, the concentration of tethers added to the well comprises between about 100 nM and 1 μM, 500 nM and 2 μM, 1 μM and 10 μM, or 5 μM and 50 μM.

In some embodiments, the method further comprises a step of measuring a property indicative of the translocation of the first and second nucleic acids of the pair, obtaining data indicative of the measured property, and determining a characteristic of the double stranded nucleic acid complex based upon the obtained data of both the first and second nucleic acids.

In some embodiments, the method further comprises detecting a signal corresponding to ion flow through the nanopore to detect polynucleotides of the first and second nucleic acids translocating through the pore; identifying a signal corresponding to translocation of the first nucleic acid of the pair and a sequential signal corresponding to separate translocation of the second nucleic acid of the pair; and analyzing the identified signals, thereby sequencing the double stranded nucleic acid complex.

In some aspects, the disclosure provides a system comprising a double-stranded nucleic acid complex each complex comprising a pair of non-covalently bound single stranded nucleic acids, each single stranded nucleic acid of the pair comprising an adaptor having a leader to a nanopore disposed in a membrane, wherein a potential is applied across the membrane to promote translocation of the single stranded nucleic acids through the nanopore, and wherein the system is configured such that the likelihood of nucleic acids of a pair translocating through the nanopore sequentially is greater than the likelihood of nucleic acids from different pairs of non-covalently bound single stranded nucleic acids translocating through the nanopore sequentially.

In some aspects, the disclosure provides a system comprising a double-stranded nucleic acid complex each complex comprising a pair of non-covalently bound single stranded nucleic acids, each single stranded nucleic acid of the pair comprising an adaptor having a leader to a nanopore disposed in a membrane, wherein a potential is applied across the membrane to promote translocation of the single stranded nucleic acids through the nanopore, and wherein the membrane comprises a plurality of tethers configured and arranged to promote sequential translocation of members of the pairs of non-covalently bound single stranded nucleic acids through the nanopore at a follow-on read frequency of at least 10 percent.

In some embodiments, the first nucleic acid and the second nucleic acid of the pair are each DNA or RNA. In some embodiments, the first nucleic acid and second nucleic acid of the pair are complementary to one another.

In some embodiments, the adaptor of a first single stranded nucleic acid of the pair is positioned on the 5′ end of the first single stranded nucleic acid. In some embodiments, the adaptor of a second single stranded nucleic acid of the pair is positioned on the 5′ end of the second single stranded nucleic acid.

In some embodiments, each leader comprises one or more poly-dT section. In some embodiments, each leader comprises two or more poly-dT sections, optionally wherein each of the poly-dT sections are non-contiguous.

In some embodiments, each adaptor further comprises one or more spacers. In some embodiments, each of the one or more spacers are selected from an iSp3C spacer, iSpC9 spacer, and iSpC18 spacer.

In some embodiments, each adaptor further comprises one or more modified nucleotides. In some embodiments, the modified nucleotides are 2′-O-Methyl (2′OMe) modified nucleotides.

In some embodiments, the nanopore is a protein nanopore. In some embodiments, the nanopore is a CsgG nanopore.

In some embodiments, each of the tethers is a lipid, fatty acid, sterol, carbon nanotube, polypeptide, protein or amino acid. In some embodiments, each of the tethers comprises tocopherol. In some embodiments, each of the tethers comprises octyl-tocopherol.

In some embodiments, the likelihood of nucleic acids of a pair translocating through the nanopore sequentially is at least 15%, 20%, 25%, or 30% greater than the likelihood of nucleic acids from different pairs of non-covalently bound single stranded nucleic acids translocating through the nanopore sequentially.

In some embodiments, each tether comprises a hydrophobic anchor and a tether linker comprising a polynucleotide coupled to the hydrophobic anchor, wherein each adaptor comprises a polynucleotide at least a portion of which hybridises with a corresponding portion of the tether linker to form a section of double stranded polynucleotide having a length of about 24 to 30 base pairs.

In some aspects, the disclosure provides a method for sequentially translocating two non-covalently bound molecules through a nanopore, the method comprising contacting a double stranded nucleic acid complex comprising a pair of non-covalently bound single stranded nucleic acids, each single stranded nucleic acid of the pair comprising an adaptor having a leader to a nanopore disposed in a membrane comprising a plurality of tethers, said membrane being contained in a well, wherein the concentration of tethers added to the well is at least 1 μM; and applying a potential to the membrane, wherein after application of the potential, the first single stranded nucleic acid translocates through the nanopore, and as the first single stranded nucleic acid translocates, reversibly binding the second single stranded nucleic acid to at least one of the tethers that is present on the membrane, and after the first single stranded nucleic acid of the pair has completely translocated through the nanopore the second single stranded nucleic acid of the pair translocates through the nanopore.

In some aspects, the disclosure provides a method for sequentially translocating two non-covalently bound molecules through a nanopore, the method comprising providing a double stranded nucleic acid complex comprising a pair of non-covalently bound single stranded nucleic acids, each single stranded nucleic acid of the pair comprising an adaptor having a leader; contacting the double-stranded nucleic acid complex of (i) to a nanopore disposed in a membrane comprising a plurality of tethers, said membrane being contained in a well, wherein the concentration of tethers added the well is at least 1 μM, under conditions that promote translocation of a first single stranded nucleic acid of the pair through the nanopore; reversibly binding the second single stranded nucleic acid to at least one of the tethers that is present on the membrane; and translocating the second single stranded nucleic acid of the pair through the nanopore after the first single stranded nucleic acid of the pair has completely translocated through the nanopore.

In some embodiments, the first nucleic acid and the second nucleic acid of the pair are each DNA or RNA. In some embodiments, the first nucleic acid and second nucleic acid of the pair are complementary to one another.

In some embodiments, each leader comprises one or more poly-dT section. In some embodiments, each leader comprises two or more poly-dT sections, wherein each of the poly-dT sections are non-contiguous.

In some embodiments, each adaptor further comprises one or more spacers. In some embodiments, each of the one or more spacers are selected from an iSp3C spacer, iSpC9 spacer, and iSpC18 spacer.

In some embodiments, each adaptor further comprises one or more modified nucleotides. In some embodiments, the modified nucleotides are 2′-O-Methyl (2′OMe) modified nucleotides.

In some embodiments, the nanopore is a protein nanopore. In some embodiments, the nanopore is a CsgG nanopore.

In some embodiments, each of the tethers is a lipid, fatty acid, sterol, carbon nanotube, polypeptide, protein or amino acid. In some embodiments, each of the tethers comprises tocopherol, optionally wherein each of the tethers comprises octyl-tocopherol. In some embodiments, the concentration of tethers added the well comprises between about 1 μM and 5 μM. 2 μM and 20 μM, or 10 μM and 50 μM.

In some embodiments, the conditions that promote translocation of a first single stranded nucleic acid of the pair the nanopore comprise applying a potential across the membrane.

In some embodiments, translocating the second single stranded nucleic acid of the pair through the nanopore comprises capture of the leader of the second single stranded nucleic acid by the nanopore.

In some embodiments, the second single stranded nucleic acid of the pair translocates through the nanopore immediately after the first single stranded nucleic acid of the pair.

In some embodiments, one or more nucleic acids that are not part of the complex translocate through the nanopore prior to the second single stranded nucleic acid of the pair translocating through the nanopore.

In some embodiments, the first single stranded nucleic acid and the second single stranded nucleic acid are no longer non-covalently bound after the first single stranded nucleic acid completely translocates through the nanopore.

In some embodiments, the method further comprises measuring a property indicative of the translocation of the first and second nucleic acids of the pair, obtaining data indicative of the measured property, and determining a characteristic of the double stranded nucleic acid complex based upon the obtained data of both the first and second nucleic acids.

In some embodiments, the method further comprises detecting a signal corresponding to ion flow through the nanopore to detect polynucleotides of the first and second nucleic acids translocating through the pore; identifying a signal corresponding to translocation of the first nucleic acid of the pair and a sequential signal corresponding to separate translocation of the second nucleic acid of the pair; and analyzing the identified signals, thereby sequencing the double stranded nucleic acid complex.

In some aspects, the disclosure provides a double stranded nucleic acid complex comprising a first single stranded nucleic acid comprising a first template nucleic acid section, and a first adaptor, wherein the first adaptor comprises a leader sequence comprising at least two non-continuous poly-dT sections, wherein the first single stranded nucleic acid is non-covalently bound to a second single stranded nucleic acid comprising a second template nucleic acid section that is complementary to the first template nucleic acid section, and a second adaptor, wherein the second adaptor comprises a leader sequence comprising at least two non-continuous poly-dT sections; and a tether.

In some embodiments, the first template nucleic acid section and/or the second template nucleic acid section is DNA or RNA.

In some embodiments, each leader comprises three or more non-continuous poly-dT sections.

In some embodiments, each adaptor further comprises one or more spacers. In some embodiments, each of the one or more spacers are selected from an iSp3C spacer, iSpC9 spacer, and iSpC18 spacer.

In some embodiments, each adaptor further comprises one or more modified nucleotides. In some embodiments, the modified nucleotides are 2′-O-Methyl (2′OMe) modified nucleotides.

In some embodiments, each of the tethers is a lipid, fatty acid, sterol, carbon nanotube, polypeptide, protein or amino acid. In some embodiments, each of the tethers comprises tocopherol. In some embodiments, each of the tethers comprises octyl-tocopherol. In some aspects, the disclosure provides a system for nucleic acid sequencing comprising a well comprising a nanopore disposed in a membrane; a plurality of tethers, wherein the concentration of the plurality of tethers added to the well is at least 100 nM; a double stranded nucleic acid molecule comprising a first strand hybridized to a complementary second strand, each strand comprising a leader sequence comprising at least two non-continuous poly-dT sections.

### DESCRIPTION OF THE FIGURES

FIG. 1 shows increased strand capture in the pore when using a leader comprising poly-dT compared to a leader comprising only iSpC3 spacer molecules, as detailed in Example 2.

FIG. 2 shows follow-on percentages obtained using four different hybridisation lengths, as detailed in Example 3. Follow-on classes are presented in each bar from top to bottom in accordance with the key.

### DETAILED DESCRIPTION

Aspects of the disclosure relate to compositions and methods for characterizing nucleic acids using a nanopore. The disclosure is based, in part, on methods for increasing follow-on sequencing of nucleic acid strands. As used herein, “follow-on” or “follow-on event” refers to the translocation of two complementary nucleic acid strands of a double stranded nucleic acid molecule through a nanopore in a sequential (e.g., one strand after the other) manner. In some embodiments, follow-on comprises the two complementary nucleic acid strands (e.g., a pair of strands) of a double stranded nucleic acid molecule translocate through a nanopore in immediate succession (e.g., no single stranded nucleic acids of other molecules pass through the pore before the two nucleic acid strands of the pair. In some embodiments, follow-on comprises one or more nucleic acids (e.g., 1, 2, 3, 4, 5, etc.) that are not part of the complementary nucleic acid pair (e.g., complementary strands of a double stranded nucleic acid molecule) translocate through the pore between the translocation of the first nucleic acid and the second nucleic acid of the pair through the nanopore. In some embodiments, follow-on comprises less than 10 nucleic acids (e.g., 10, 9, 8, 7, 6, 5, 4, 3, 2, or 1) that are not part of the complementary nucleic acid pair (e.g., complementary strands of a double stranded nucleic acid molecule) translocate through the pore between the translocation of the first nucleic acid and the second nucleic acid of the pair through the nanopore. In some embodiments, methods and systems described herein surprisingly increase follow-on events during nanopore sequencing to more than about 10%, 15%, 25%, or 30%. In some embodiments, methods and systems described herein surprisingly increase follow-on events during nanopore sequencing to more than 30%. In some embodiments, methods and systems described herein surprisingly increase follow-on events during nanopore sequencing to more than about 35%, 40%, 45%, 50%, 55%, 60%, 65%, 70%, or 75%. Without wishing to be bound by any particular theory, increased follow-on events mediated by compositions, systems, and methods described herein improve sequencing quality, for example as measured by Q-score.

In some embodiments, the methods described herein further comprise performing an alignment to identify pairs of single stranded nucleic acids of a double stranded nucleic acid complex, wherein the alignment is made between the sequences of candidate pairs or between the sequences of candidate pairs and a reference sequence. In one embodiment, in the event of a single stranded nucleic acid being identified as pairing with more than one other nucleic acid strand, the two strands that translocated the nanopore closest in time to one another may be determined to be the actual pair. In one embodiment, the method may therefore further comprise measuring the time of translocation of single stranded nucleic acids to determine the order of translocation and time between translocations.

### Nucleic Acids

Aspects of the disclosure relate to compositions and methods for sequencing nucleic acids. In some embodiments, the nucleic acids are double stranded. In some embodiments, double stranded nucleic acids comprise a pair of non-covalently bound single stranded nucleic acids.

As used herein, the term “non-covalently bound molecule” refers to a molecule comprising a first a member and a second member, wherein the first member and the second member are associated with each other by means of non-covalent attachment and can be separated from each other as individual entities. The separation and binding process between the first member and the second member are reversible. Examples of means of non-covalent attachment include, but are not limited to complementary base-pairing, ionic interaction, hydrophobic interaction, and/or Van der Waals' interaction.

In some embodiments, the non-covalently bound molecules comprise complementary polynucleotide strands. The length of a region of complementarity between two polynucleotide strands (e.g., region over which complementary base pairing between the strands occurs) may vary. In some embodiments, two polynucleotide strands are at least 50%, 60%, 70%, 80%, 90%, 95%, 99%, or 99% complementary over the entire length of the two polynucleotide strands. In some embodiments, two polynucleotide strands are 100% complementary over the entire length of the two polynucleotide strands. In some embodiments, two polynucleotide strands are at least 50%, 60%, 70%, 80%, 90%, 95%, 99%, or 99% complementary over the length of the shorter of the two polynucleotide strands. In some embodiments, two polynucleotide strands are 100% complementary over the length of the shorter of the two polynucleotide strands.

In some embodiments, the pair of non-covalently bound molecules comprise a target nucleic acid (e.g., a target double stranded polynucleotide) coupled to an adaptor. Adaptors are described generally throughout the specification and in detail in the section entitled ‘Adaptor’ below.

It should be noted that the adaptors described herein can be attached to either or both ends of a double stranded polynucleotide (e.g., the 5′ end of each polynucleotide strand, the 3′ end of each polynucleotide strand, or both the 5′ and 3′ ends of each polynucleotide strand). In some embodiments, the same adaptors are attached to both ends of a double stranded polynucleotide. In some embodiments, different adaptors can be attached to the ends of a double stranded polynucleotide. Attachment of different adaptors to the ends of double stranded polynucleotides can be achieved, for example, by mixing two or more populations of different adaptors together with the double stranded polynucleotides. Typically, a mixture of double stranded polynucleotides attached with different adaptors is formed, but there are also methods to achieve a desired hetero-adapter mixture (e.g., through purification or by controlling the attachment of adaptors to the ends of double stranded polynucleotides).

The polynucleotide can be a nucleic acid, such as deoxyribonucleic acid (DNA) or ribonucleic acid (RNA). The polynucleotide can comprise one strand of RNA hybridized to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains. The PNA backbone is composed of repeating N-(2-aminoethyl)-glycine units linked by peptide bonds. The GNA backbone is composed of repeating glycol units linked by phosphodiester bonds. The TNA backbone is composed of repeating threose sugars linked together by phosphodiester bonds. LNA is formed from ribonucleotides as discussed above having an extra bridge connecting the 2′ oxygen and 4′ carbon in the ribose moiety.

The polynucleotide is preferably DNA, RNA or a DNA or RNA hybrid, most preferably DNA. The target polynucleotide may be double stranded. The target polynucleotide may comprise single stranded regions and regions with other structures, such as hairpin loops, triplexes and/or quadruplexes. The DNA/RNA hybrid may comprise DNA and RNA on the same strand. Preferably, the DNA/RNA hybrid comprises one DNA strand hybridized to an RNA strand.

In some embodiments, the target polynucleotide does not comprise a hairpin structure or any covalent linkage to connect a template and a complement. In some embodiments, the target polynucleotide (e.g., template) and polynucleotide complementary to the target polynucleotide (e.g., complement) are not linked by a bridging moiety, such as a hairpin loop. However, in some embodiments, as a single strand (e.g., template or complement) translocates through a nanopore, the strand itself can form a hairpin structure due to the interaction of the adaptors on its both ends. Such adaptor design can be beneficial for characterizing a long polynucleotide, e.g., by maintaining the other end of the strand close to the nanopore.

Each nucleic acid strand of a complex (e.g., a target polynucleotide strand or its complement) can be any length. For example, the polynucleotides can be at least 10, at least 50, at least 100, at least 150, at least 200, at least 250, at least 300, at least 400 or at least 500 nucleotides or nucleotide pairs in length. The target polynucleotide can be 1000 or more nucleotides or nucleotide pairs, 5000 or more nucleotides or nucleotide pairs in length or 100000 or more nucleotides or nucleotide pairs in length or 500,000 or more nucleotides or nucleotide pairs in length, or 1,000,000 or more nucleotides or nucleotide pairs in length, 10,000,000 or more nucleotides or nucleotide pairs in length, or 100,000,000 or more nucleotides or nucleotide pairs in length, or 200,000,000 or more nucleotides or nucleotide pairs in length, or the entire length of a chromosome. The target polynucleotide may be an oligonucleotide. Oligonucleotides are short nucleotide polymers which typically have 50 or fewer nucleotides, such 40 or fewer, 30 or fewer, 20 or fewer, 10 or fewer or 5 or fewer nucleotides. The target oligonucleotide is preferably from about 15 to about 30 nucleotides in length, such as from about 20 to about 25 nucleotides in length. For example, the oligonucleotide can be about 15, about 16, about 17, about 18, about 19, about 20, about 21, about 22, about 23, about 24, about 25, about 26, about 27, about 28, about 29 or about 30 nucleotides in length.

The target polynucleotide may be a fragment of a longer target polynucleotide. In this embodiment, the longer target polynucleotide is typically fragmented into multiple, such as two or more, shorter target polynucleotides. The method of the invention may be used to sequence one or more, such as 2, 3, 4, 5 or more of those shorter target polynucleotides.

In some embodiments, the method of various aspects described herein may be used to sample multiple target polynucleotides, such as 2, 3, 4 or 5 to 10, 15, 20 or more polynucleotides, within a sample.

In some embodiments, the method of various aspects described herein may be used to sequence polynucleotides that are present in double stranded form in a sample.

In some embodiments, a double stranded polynucleotide can have an adaptor to its 3′ end or 5′ end. Such a configuration may also be referred to herein as a double stranded nucleic acid complex.

In some embodiments, a double stranded polynucleotide can have an adaptor attached to the 3′ end of each polynucleotide strand or to the 5′ end of each polynucleotide strand. Such a configuration may also be referred to herein as a double stranded nucleic acid complex.

The target polynucleotide is typically present in a sample comprising multiple copies of the target polynucleotide and/or in a sample comprising multiple different polynucleotides. In some embodiments, the method of any aspects described herein may comprise determining the sequence of one or more target polynucleotides in a sample. The method may comprise contacting the pore with two or more double stranded polynucleotides. For example, the method may comprise contacting the pore with a sample in which substantially all the double stranded polynucleotides have a single stranded leader sequence on each of their two strands. In some embodiments, the double stranded polynucleotides are coupled to each other only via complementary base pairing. In these embodiments, the double stranded polynucleotides can have four free ends, wherein a free end is the end of a polynucleotide strand. The end of the polynucleotide strand may be single stranded, e.g., a single stranded overhang, or base paired to another polynucleotide strand. In some embodiments, the two strands of the double stranded polynucleotides being sequenced are not covalently attached (e.g., no hairpin or other covalent attachment). However, a moiety that does not bridge the template and complement polynucleotides may be added to one or more of the free ends.

### Sample

Aspects of the disclosure relate to sequencing one or more analytes (e.g., target polynucleotides) present in a sample (e.g., a sample obtained from a subject, e.g., a human subject). The analytes may include proteins, peptides, molecules, polypeptides, polynucleotides, etc. The sample may be any suitable sample. The sample may be a biological sample. Any embodiment of the methods described herein may be carried out in vitro on a sample obtained from or extracted from any organism or microorganism. The organism or microorganism is typically archaean, prokaryotic or eukaryotic and typically belongs to one of the five kingdoms: plantae, animalia, fungi, monera and protista. In some embodiments, the methods of various aspects described herein may be carried out in vitro on a sample obtained from or extracted from any virus.

The sample is preferably a fluid sample. The sample typically comprises a body fluid. The body fluid may be obtained from a human or animal. The human or animal may have, be suspected of having or be at risk of a disease. The sample may be urine, lymph, saliva, mucus, seminal fluid or amniotic fluid, but is preferably whole blood, plasma or serum. Typically, the sample is human in origin, but alternatively it may be from another mammal such as from commercially farmed animals such as horses, cattle, sheep or pigs or may alternatively be pets such as cats or dogs.

Alternatively a sample of plant origin is typically obtained from a commercial crop, such as a cereal, legume, fruit or vegetable, for example wheat, barley, oats, canola, maize, soya, rice, bananas, apples, tomatoes, potatoes, grapes, tobacco, beans, lentils, sugar cane, cocoa, cotton, tea or coffee.

The sample may be a non-biological sample. The non-biological sample is preferably a fluid sample. Examples of non-biological samples include surgical fluids, water such as drinking water, sea water or river water, and reagents for laboratory tests.

The sample may be processed prior to being assayed, for example by centrifugation or by passage through a membrane that filters out unwanted molecules or cells, such as red blood cells. The sample may be measured immediately upon being taken. The sample may also be typically stored prior to assay, preferably below −70° C.

In some embodiments, the sample may comprise genomic DNA. The genomic DNA may be fragmented or any of the methods described herein may further comprise fragmenting the genomic DNA. The DNA may be fragmented by any suitable method. For example, methods of fragmenting DNA are known in the art. Such methods may use a transposase, such as a MuA transposase or a commercially available G-tube.

### Leader

Aspects of the disclosure relate to double stranded nucleic acid complexes comprising a first nucleic acid strand and a second nucleic acid strand, each strand comprising a leader (also referred to as a leader sequence). The leader sequence typically comprises a polymer. The polymer is preferably negatively charged. The polymer is preferably a polynucleotide, such as DNA or RNA, a modified polynucleotide (such as a basic DNA), PNA, LNA, polyethylene glycol (PEG) or a polypeptide. The leader preferably comprises a polynucleotide and more preferably comprises a single stranded polynucleotide.

The leader sequence can be any length, but is typically 10 to 150 nucleotides in length, such as from 20 to 150 nucleotides in length. The length of the leader typically depends on the transmembrane pore used in the method.

The disclosure is based, in part, on the recognition that leader sequences that are rigid or stiffened (e.g., relative to previously used leader sequences) provide, in some embodiments, enhanced follow-on during strand sequencing of double stranded nucleic acids. In some embodiments, a leader (e.g., stiffened or rigid leader) comprises one or more poly dT sections. In some embodiments, a leader (e.g., stiffened or rigid leader) comprises 2, 3, 4, 5, 6, 7, 8, 9, or poly dT sections. The length of each poly dT section may vary. In some embodiments, each 10 poly dT section ranges from about 2 to about 15 dT nucleotides (e.g., 2, 3, 4, 5, 6, 7, 8, 9, 10, 11, 12, 13, 14, or 15 nucleotides) in length. In some embodiments, each poly dT section ranges from about 2 to about 30 dT nucleotides (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotides) in length. In some embodiments, each poly dT section ranges from about 5 to about 10 dT nucleotides (e.g., 5, 6, 7, 8, 9 or 10 nucleotides) in length.

In some embodiments, the poly dT sections are non-continuous (e.g., each poly dT section is present along the same phosphate-based backbone but is separated by one or more nucleotides that does not comprise a dT nucleoside). The length of leader sequence that separates the poly dT sections may vary. In some embodiments, the length of non poly dT leader sequence ranges from about 1 to about 5 (e.g., 1, 2, 3, 4, or 5) nucleotides in length. Each of the non poly dT nucleotides may be selected from A, G, or C, or modified versions thereof. In some embodiments, a leader (e.g., stiffened of rigid leader) comprises less than 1, 2, 3, 4, 5, 6, 7, 8, 9, or 10 nucleotides that are not dT nucleotides.

The disclosure is based, in part, on leaders that comprise fewer spacer molecules relative to previously used leaders. Without wishing to be bound by theory, a reduction in spacer molecules is thought to increase the rigidity (e.g., stiffness) of the leader and contribute to improved follow-on during nanopore sequencing. In some embodiments, a leader comprises 1, 2, 3, 4, 5, 6, 7, 8, 9, or 10 spacer molecules. In some embodiments, each spacer molecule is individually selected from an iSpC3, iSpC9, and iSpC18 molecule, for example as described by Integrated DNA Technologies (also referred to as C3, iSp9, and iSp18 spacer molecules).

In some embodiments, a spacer molecule may comprise a nitroindole, an inosine, an acridine, a 2-aminopurine, a 2-6-diaminopurine, a 5-bromo-deoxyuridine, an inverted thymidine, an inverted dideoxy-thymidine, a dideoxy-cytidine (ddC), a 5-methylcytidine, a 5-hydroxymethylcytidine, a 2′-O-Methyl RNA base, an Iso-deoxycytidine (Iso-dC), an Iso-deoxyguanosine (Iso-dG), a C3 (OC₃H₆OPO₃) group, a photo-cleavable (PC) [OC₃H₆—C(O)NHCH₂-C₆H₃NO₂—CH(CH₃)OPO₃] group, a hexandiol group, a spacer 9 (iSp9) [(OCH₂CH₂)₃OPO₃] group, or a spacer 18 (iSp18) [(OCH₂CH₂)₆OPO₃] group.

The leader sequence preferentially threads into the transmembrane pore and thereby facilitates the movement of polynucleotide through the pore. The leader sequence can also be used to link the polynucleotide to the one or more anchors as discussed herein.

Typically, a leader sequence is present at one end of the target polynucleotide and at one end of the polynucleotide complementary to the target polynucleotide. Leader sequences may be present at the 5′ end of the target polynucleotide and at the 5′ end of the complement of the target polynucleotide. Alternatively, leader sequence may be present at the 3′ end of the target polynucleotide and at the 3′ end of the complement of the target polynucleotide. A leader sequence may be present at the 5′ end of the target polynucleotide and at the 3′ end of the complementary polynucleotide, or vice versa. In these latter embodiments, two different polynucleotide binding proteins (e.g., polynucleotide unwinding enzymes) are typically used, wherein a first polynucleotide binding protein (e.g., polynucleotide unwinding enzyme) moves along the polynucleotide in a 5′ to 3′ direction and a second polynucleotide binding protein (e.g., polynucleotide unwinding enzyme) moves along the polynucleotide in a 3′ to 5′ direction.

The leader sequence may be attached to the double stranded polynucleotide by any suitable method. For example, the leader sequence may be ligated to the target polynucleotide and/or to the complement thereof. Alternatively, the leader sequence may be generated by digesting one strand of the double stranded polynucleotide to produce a single stranded overhang on the other strand.

A polynucleotide binding protein (e.g., polynucleotide unwinding enzyme) may be bound to the leader sequence prior to its attachment to the target polynucleotide or complement thereof. A polynucleotide binding protein (e.g., polynucleotide unwinding enzyme) may be bound to a leader sequence present in the double stranded polynucleotide. The activity of the polynucleotide binding protein (e.g., polynucleotide unwinding enzyme) bound to the leader sequence may be stalled until the polynucleotide contacts the transmembrane pore. Methods of stalling polynucleotide binding protein (e.g., polynucleotide unwinding enzyme)s are known in the art, for example in WO 2014/135838.

### Adaptor

The leader sequence may be present in an adaptor. In some embodiments, an adaptor comprises a double stranded region (e.g., a duplex stem) and at least one single stranded region. At least one of the single stranded regions may be a leader sequence. The adaptor may comprise at least one non-polynucleotide region. The adaptors attached to the two ends of the target double stranded polynucleotide may be the same or different. Preferably, the adaptors in the pair are the same.

The leader sequence is preferably present in a first single stranded region at the 5′ end (or 3′ end) of one strand of the adaptor. A second single stranded region may be present at the 3′ end (or 5′ end) of the other strand of the adaptor. The first and second single stranded regions of the adaptor are not complementary. In this embodiment, the adaptor may be referred to as a Y adaptor.

A Y adaptor typically comprises (a) a double stranded region (e.g., a duplex stem) and (b) a single stranded region or a region that is not complementary at the other end. A Y adaptor may be described as having an overhang if it comprises a single stranded region. The presence of a non-complementary region in the Y adaptor gives the adaptor its Y shape since the two strands typically do not hybridise to each other unlike the double stranded portion. The Y adaptor may comprise one or more anchors.

In some embodiments, a Y adaptor comprises a leader sequence which preferentially threads into the pore. In some embodiments, a Y adaptor may be attached to a polynucleotide using any method known in the art. For example, one or both of the adaptors may be ligated using a ligase, such as T4 DNA ligase, E. coli DNA ligase, Taq DNA ligase, Tma DNA ligase and 9° N DNA ligase.

In some embodiments, the double stranded polynucleotides in a sample are modified so that they comprise Y adaptors at both ends. Any manner of modification can be used. The method may comprise modifying the double stranded target polynucleotide by adding the adaptors.

The double stranded polynucleotide may be provided with adaptors, such as Y adaptors, or anchors (e.g., tethers) by contacting the polynucleotide with a MuA transposase and a population of double stranded MuA substrates. The transposase fragments the double stranded polynucleotide and ligates MuA substrates to one or both ends of the fragments. This produces a plurality of modified double stranded polynucleotides comprising an adaptor or anchor. The modified double stranded polynucleotides may then be investigated using the method of the invention. These MuA based methods are disclosed in WO 2015/022544 and WO 2016/059363. They are also discussed in detail in WO2015/150786.

An adaptor may further comprise an anchor to tether the double stranded polynucleotide comprising the target polynucleotide and/or its complement to the membrane comprising the pore. i.e. the adaptor may further comprise a membrane-tether. The anchor is preferably attached to the single stranded region that is not the leader sequence.

In some embodiments, the adaptor has a polynucleotide binding protein bound to it, for example bound to the leader. Suitable methods for loading a polynucleotide binding protein onto a polynucleotide adaptor are described in WO 2020/234612, which is incorporated herein by reference in its entirety.

The polynucleotide binding protein (e.g., polynucleotide unwinding enzyme) may be bound to the leader sequence in the adaptor, or the polynucleotide binding protein (e.g., polynucleotide unwinding enzyme) may be added after the adaptor has been attached to the double stranded polynucleotide. The activity of the polynucleotide binding protein (e.g., polynucleotide unwinding enzyme) bound to the leader sequence may be stalled until the polynucleotide contacts the transmembrane pore.

The leader sequence or adaptor may be attached to the double stranded polynucleotide by any suitable method. For example, the leader sequence may be ligated to the target polynucleotide and/or to the complement thereof or the adaptor may be ligated to the double stranded polynucleotide.

In some embodiments, a double stranded barcode sequence may be ligated to one or both ends of the target double stranded polynucleotide. The barcode sequence may be added to the double stranded polynucleotide before the leader sequence or adaptor is added. For example, the barcode sequence may be located between the end of the target double stranded polynucleotide and the adaptor. In some embodiments, the barcode sequence is comprised in the adaptor.

A unique barcode sequence may be attached, for example ligated, to each double stranded polynucleotide in a sample. The barcode sequence may be used to identify signals corresponding to sequential translocation through the pore of the target polynucleotide and the polynucleotide complementary to the target polynucleotide.

In some embodiments, an adaptor (e.g., section of adaptor that does not comprise the leader) described herein can comprise one or more spacers to prevent pre-bound polynucleotide binding protein (e.g., a polynucleotide unwinding enzyme) from moving along and unwinding a double stranded polynucleotide. These spacers prevent further movement of the polynucleotide binding protein (e.g., a polynucleotide unwinding enzyme) until the polynucleotide binding protein (e.g., a polynucleotide unwinding enzyme) is located at the pore and a potential difference is applied across the pore. The additional force provided by the potential difference pushes the polynucleotide binding protein (e.g., a polynucleotide unwinding enzyme) over the spacers and allows it to unwind and control movement of the polynucleotide through the nanopore. Thus movement by the polynucleotide binding protein (e.g., a polynucleotide unwinding enzyme) typically only occurs when the polynucleotide is in the nanopore and not before. Examples of spacers and methods for preventing pre-bound polynucleotide binding protein (e.g., a polynucleotide unwinding enzyme) from moving along and unwinding a double stranded polynucleotide until the polynucleotide is in a nanopore are described, for example, in WO2015/110813, the contents of which are incorporated herein by reference in their entireties.

Further examples of adaptors suitable for use in methods of characterising double-stranded polynucleotides are described in WO 2018/100370 and WO 2020/234612, the contents of which are incorporated herein by reference in their entireties.

### Tethers

Aspects of the disclosure relate to methods and systems for improving follow-on that comprise tethering agents (also referred to as anchors, tethers, or membrane-tethers). One or more tethers may be used to couple a double stranded nucleic acid complex (e.g., a double stranded target polynucleotide where each strand of the polynucleotide comprises a leader or adaptor) to the membrane. Typically, one or more tethers are attached to each strand of the target polynucleotide. In some embodiments, the tether is part of an adaptor. Examples of tethers and methods of attaching tethers to adapters are disclosed in WO 2012/164270 and WO 2015/150786, the contents of which are incorporated herein by reference in their entireties.

If the membrane is an amphiphilic layer, such as a triblock copolymer membrane, the one or more tethers preferably comprises a polypeptide anchor and/or a hydrophobic anchor that can be inserted into the membrane. The hydrophobic anchor preferably comprises a lipid, fatty acid, sterol, carbon nanotube, polypeptide, protein or amino acid, for example cholesterol, palmitate or tocopherol. In preferred embodiments, the one or more tethers are not connected (e.g., bound to) the nanopore.

In some embodiments, tethering agents are part of a tethering complex, and the tethering complex is concentrated in a region of an amphiphilic layer. Methods of concentrating a tethering complex in a region of an amphiphilic layer are described in PCT/GB2020/053104 (corresponding to International Publication WO 2021/111139), incorporated herein by reference in its entirety.

The components of the membrane, such as the amphiphilic molecules, copolymer or lipids, may be chemically-modified or functionalized to form the one or more anchors. Examples of suitable chemical modifications and suitable ways of functionalizing the components of the membrane are discussed in more detail below. Any proportion of the membrane components may be functionalized, for example at least 0.01%, at least 0.1%, at least 1%, at least 10%, at least 25%, at least 50% or 100%.

In some embodiments, the one or more anchors preferably comprise one or more linkers. The one or more anchors may comprise one or more, such as 2, 3, 4 or more, linkers. In some embodiments, each linker is selected from an iSpC3, iSpC9 (iSp9), and iSpC18 (iSp18) molecule, for example as described by Integrated DNA Technologies. Additional examples of linkers include, but are not limited to, polymers, such as polynucleotides, polyethylene glycols (PEGs), polysaccharides and polypeptides. These linkers may be linear, branched or circular.

For instance, the linker may be a circular polynucleotide. The target polynucleotide may hybridize to a complementary sequence on the circular polynucleotide linker.

In some embodiments, the one or more tethers or one or more linkers may comprise a component that can be cut or broken down, such as a restriction site or a photo-labile group.

Functionalized linkers and the ways in which they can couple molecules are known in the art. For instance, linkers functionalized with maleimide groups will react with and attach to cysteine residues in proteins.

Cross-linkage of polynucleotides can be avoided using a “lock and key” arrangement. Only one end of each linker may react together to form a longer linker and the other ends of the linker each react with the polynucleotide or membrane respectively. Such linkers are described in WO 2010/086602.

The coupling of a double stranded nucleic acid complex to a membrane via one or more tethers may be permanent or stable. In other words, the coupling may be such that the polynucleotide remains coupled to the membrane when interacting with the pore.

The coupling may be transient. In other words, the coupling may be such that the polynucleotide may decouple from the membrane when interacting with the pore. For polynucleotide sequencing, the transient nature of the coupling is preferred. If a permanent or stable linker is attached directly to either the 5′ or 3′ end of a polynucleotide and the linker is shorter than the distance between the membrane and the channel of the transmembrane pore, then some sequence data will be lost as the sequencing run cannot continue to the end of the polynucleotide. If the coupling is transient, then when the coupled end randomly becomes free of the membrane, then the polynucleotide can be processed to completion. The target polynucleotide and/or its complement may be transiently coupled to a membrane such as an amphiphilic layer e.g. triblock copolymer membrane or lipid membrane using cholesterol, a fatty acyl chain, or tocopherol. Any fatty acyl chain having a length of from 6 to 30 carbon atom, such as hexadecanoic acid, may be used.

In some embodiments, a tether comprises a tocopherol. Tocopherols are compounds comprising a chromane ring with a hydroxyl group, and a hydrophobic side chain. The four known forms of tocopherols, α (alpha), β (beta), γ (gamma), and δ (delta), differ in the positioning of methyl groups on the chromane ring. In some embodiments, a tether comprises a tocopherol and one or more linkers, for example an iSpC3 linker, iSpC8 linker, iSpC9 molecule, etc. In some embodiments, a tether comprises a tocopherol and an iSpC8 linker (also referred to as octyl-tocopherol).

The disclosure is based, in part, on the recognition that increasing concentrations of tether in a well comprising a nanopore disposed in a membrane improves follow-on event frequency. The concentration of tether (e.g., concentration of tether added to a well comprising a nanopore disposed in a membrane) may vary. In some embodiments, a concentration between about 100 nM and 500 nM, 250 nM and 800 nM, 400 nM and 1 μM, 600 nM and 1.5 μM, 1.0 μM and 2.5 μM. 2.0 μM and 4.0 μM, or 3.0 and 5.0 μM is added to the well. In some embodiments, more than 5.0 μM (e.g., 8 μM, 10 μM, 15 μM) is added to the well. As described in the Example, it has been observed that increasing the concentration of tether surprisingly increases follow-on events during nanopore sequencing. This observation is surprising because increased concentrations of tether (e.g., adding >50 nM tether) in a well comprising a nanopore disposed in a membrane were previously thought to undesirably contribute to pore blockage.

In some embodiments, the one or more tethers are mixed with the double stranded nucleic acid complex before delivery to the membrane. In some embodiments, the one or more tethers are contacted with the membrane and subsequently contacted with the double stranded nucleic acid complex.

According to some embodiments, the one or more tethers (e.g., octyl-tocopherol tethers) may be used to couple a double stranded nucleic acid complex to the membrane when a strand of the complex is attached to a leader sequence (e.g., a stiffened or rigid leader) which preferentially threads into the pore.

In some embodiments, the double-stranded nucleic acid complex comprises an adaptor, and the complex is coupled to the membrane via an interaction between the adapter and the tether.

In some embodiments, the tether comprises a hydrophobic anchor and a linker (also referred to as a tether linker) coupled to the hydrophobic anchor, the linker comprising a polynucleotide. In some embodiments, the hydrophobic anchor comprises a tocopherol. In some embodiments, the hydrophobic anchor comprises octyl-tocopherol.

In some embodiments, the hydrophobic anchor and the tether linker are joined to one another via one or more spacer molecules (for example, 1, 2, 3, 4, 5, 6, 7, 8, 9 or 10 spacer molecules) such as described herein. In some embodiments, the one or more spacer molecules are an iSpC3 spacer molecule, an iSp9 spacer molecule, or an iSp18 spacer molecule.

In one embodiment, the tether linker comprises a polynucleotide having a length of about 24 to about 30 nucleotides.

In one embodiment, the tether linker comprises a polynucleotide having a length of about 25 to about 30 nucleotides.

In one embodiment, the tether linker comprises a polynucleotide having a length of 24, 25, 26, 27, 28, 29 or 30 nucleotides.

In one embodiment, the tether linker comprises a polynucleotide having a length of 24, 25 or 26 nucleotides.

In one embodiment, the tether linker comprises a polynucleotide having a length of 25 nucleotides.

In some embodiments, the adaptor comprises a polynucleotide at least a portion of which hybridises with a corresponding portion of the tether linker to form a section of double stranded polynucleotide. The at least a portion of the polynucleotide comprised in the adaptor and the corresponding portion of the tether linker may be complementary to one another, for example may comprise or consist of complementary nucleic acid sequences.

In one embodiment, the section of double-stranded polynucleotide thus formed has a length of about 24 to 30 base pairs, about 25 to 30 base pairs, or about 20, 21, 22, 23, 24, 25, 26, 27, 28, 29 or 30 base pairs. In one embodiment, the section of double-stranded polynucleotide has a length of 24, 25 or 26 base pairs. In one embodiment, the section of double-stranded polynucleotide has a length of 25 base pairs.

In one embodiment, the tether linker comprises a polynucleotide at least a portion of which hybridises to a corresponding portion of the adaptor.

In one embodiment, the tether linker comprises a polynucleotide at least a portion of which hybridises to a complementary polynucleotide section of the adaptor.

In one embodiment, the tether linker comprises a polynucleotide at least a portion of which hybridises to a complementary polynucleotide section of the adaptor to form a double-stranded polynucleotide having a length of 24, 25 or 26 base pairs.

In one embodiment, the tether linker comprises a polynucleotide which hybridises to a complementary polynucleotide section of the adaptor to form a double-stranded polynucleotide having a length of 24, 25 or 26 base pairs.

In one embodiment, the tether linker comprises a polynucleotide having a length of 24, 25 or 26 nucleotides which hybridises to a complementary polynucleotide section of the adaptor to form a double-stranded polynucleotide having a length of 24, 25 or 26 base pairs.

In one embodiment, the tether linker comprises a polynucleotide at least a portion of which hybridises to a complementary polynucleotide section of the adaptor to form a double-stranded polynucleotide having a length of 25 base pairs.

In one embodiment, the tether linker comprises a polynucleotide which hybridises to a complementary polynucleotide section of the adaptor to form a double-stranded polynucleotide having a length of 25 base pairs.

In one embodiment, the tether linker comprises a polynucleotide having a length of 25 nucleotides which hybridises to a complementary polynucleotide section of the adaptor to form a double-stranded polynucleotide having a length of 25 base pairs.

The inventors have discovered that joining a tether to an adaptor via hybridisation between a tether linker and the adaptor can provide increased rates of follow-on when the length of the hybridised portion is about 24 to about 30 base pairs in length. Without wishing to be bound by theory, the inventors believe that by increasing the length of the hybridised portion, a stronger attachment is created between the tether and the adaptor, which reduces the probability that the complement strand adaptor will detach from the membrane while the template strand is passing through the pore, and so increases the probability that the complement strand adaptor remains near the pore and is captured for sequencing immediately following passage of the template strand through the pore, thus increasing rates of follow-on.

### Nanopores

A transmembrane pore is a structure that crosses the membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a gap, channel, trench or slit in the membrane along which or into which hydrated ions may flow.

Any transmembrane pore may be used in the invention. The pore may be biological or artificial. Suitable pores include, but are not limited to, protein pores, polynucleotide pores and solid state pores. The pore may be a DNA origami pore (Langecker et al., Science, 2012; 338: 932-936). The pore may be a motor protein nanopore, e.g., a nanopore that allows the translocation of a double-stranded polynucleotide. In some embodiments, the motor protein nanopore is able to unwind a double stranded polynucleotide. An exemplary motor protein nanopore includes, but is not limited to, a phi29 motor protein nanopore, e.g., as described in Wendell et al. “Translocation of double-stranded DNA through membrane-adapted phi29 motor protein nanopores” Nat Nanotechnol, 4 (2009), pp. 765-772. In some embodiments, any nanopore as described or referenced in Feng et al. “Nanopore-based fourth-generation DNA sequencing technology” Genomics, Proteomics & Bioinformatics (2015) Volume 13, Issue 1, Pages 4-16, can be used in various aspects described herein.

The transmembrane pore is preferably a transmembrane protein pore. A transmembrane protein pore is a polypeptide or a collection of polypeptides that permits hydrated ions, such as polynucleotide, to flow from one side of a membrane to the other side of the membrane. In the present invention, the transmembrane protein pore is capable of forming a pore that permits hydrated ions driven by an applied potential to flow from one side of the membrane to the other. The transmembrane protein pore preferably permits polynucleotides to flow from one side of the membrane, such as a triblock copolymer membrane, to the other. The transmembrane protein pore allows a polynucleotide, such as DNA or RNA, to be moved through the porc.

The transmembrane protein pore may be a monomer or an oligomer. The pore is preferably made up of several repeating subunits, such as at least 6, at least 7, at least 8, at least 9, at least 10, at least 11, at least 12, at least 13, at least 14, at least 15, or at least 16 subunits. The pore is preferably a hexameric, heptameric, octameric or nonameric pore. The pore may be a homo-oligomer or a hetero-oligomer.

The transmembrane protein pore typically comprises a barrel or channel through which the ions may flow. The subunits of the pore typically surround a central axis and contribute strands to a transmembrane ß barrel or channel or a transmembrane α-helix bundle or channel.

The barrel or channel of the transmembrane protein pore typically comprises amino acids that facilitate interaction with nucleotides, polynucleotides or nucleic acids. These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

Transmembrane protein pores for use in accordance with the invention can be derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A and Neisseria autotransporter lipoprotein (NalP) and other pores, such as lysenin. α-helix bundle pores comprise a barrel or channel that is formed from α-helices. Suitable α-helix bundle pores include, but are not limited to, inner membrane proteins and outer membrane proteins, such as WZA and ClyA toxin. In some embodiments, the nanopore is a CsgG nanopore.

The transmembrane pore may be derived from or based on Msp, α-hemolysin (α-HL), lysenin, CsgG, ClyA, Spl and hemolytic protein fragaceatoxin C (FraC). The transmembrane protein pore is preferably derived from CsgG, more preferably from CsgG from E. coli Str. K-12 substr. MC4100. Suitable pores derived from CsgG are disclosed in WO 2016/034591, WO 2017/149316, WO 2017/149317, WO 2017/149318, and WO 2019/002893. The transmembrane pore may be derived from lysenin. Suitable pores derived from lysenin are disclosed in WO 2013/153359.

Any of the proteins described herein, such as the transmembrane protein pores, may be modified to assist their identification or purification, for example by the addition of histidine residues (a his tag), aspartic acid residues (an asp tag), a streptavidin tag, a flag tag, a SUMO tag, a GST tag or a MBP tag, or by the addition of a signal sequence to promote their secretion from a cell where the polypeptide does not naturally contain such a sequence. An alternative to introducing a genetic tag is to chemically react a tag onto a native or engineered position on the pore or construct. An example of this would be to react a gel-shift reagent to a cysteine engineered on the outside of the pore. This has been demonstrated as a method for separating hemolysin hetero-oligomers (Chem Biol. 1997 July; 4(7):497-505). The pore may be labelled with a revealing label. The revealing label may be any suitable label which allows the pore to be detected. Suitable labels include, but are not limited to, fluorescent molecules, radioisotopes, e.g. ¹²⁵I, ³⁵S, enzymes, antibodies, antigens, polynucleotides and ligands such as biotin.

Any of the proteins described herein, such as the transmembrane protein pores, may be made synthetically or by recombinant means. For example, the pore may be synthesized by in vitro translation and transcription (IVTT). The amino acid sequence of the pore may be modified to include non-naturally occurring amino acids or to increase the stability of the protein. When a protein is produced by synthetic means, such amino acids may be introduced during production. The pore may also be altered following either synthetic or recombinant production.

Any of the proteins described herein, such as the transmembrane protein pores, can be produced using standard methods known in the art. Polynucleotide sequences encoding a pore or construct may be derived and replicated using standard methods in the art. Polynucleotide sequences encoding a pore or construct may be expressed in a bacterial host cell using standard techniques in the art. The pore may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, NY.

The pore may be produced in large scale following purification by any protein liquid chromatography system from protein producing organisms or after recombinant expression. Typical protein liquid chromatography systems include FPLC, AKTA systems, the Bio-Cad system, the Bio-Rad BioLogic system and the Gilson HPLC system.

Pores may be provided in an array, such as a nanopore array comprising a plurality of nanopores. Such arrays are described for example in WO 2014/064443, which is incorporated herein by reference in its entirety.

In some embodiments, the pore does not comprise a tag (for example a nucleic acid tag) conjugated to the pore that binds to a portion of the double-stranded nucleic acid complex. By way of example, in some embodiments the pore does not comprise a tag conjugated to the pore, such as described in WO 2018/100370; thus, in some embodiments the pore is not a tag-modified pore, such as described in WO 2018/100370.

### Membranes

Any membrane may be used in accordance with various aspects described herein Suitable membranes are well-known in the art. The membrane is preferably an amphiphilic layer or a solid state layer.

An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer. The membrane is preferably a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviors from vesicles through to laminar membranes. Membranes formed from these triblock copolymers hold several advantages over biological lipid membranes. Because the triblock copolymer is synthesized, the exact construction can be carefully controlled to provide the correct chain lengths and properties required to form membranes and to interact with pores and other proteins.

Block copolymers may also be constructed from sub-units that are not classed as lipid sub-materials; for example a hydrophobic polymer may be made from siloxane or other non-hydrocarbon-based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customize polymer-based membranes for a wide range of applications.

The membrane is most preferably one of the membranes disclosed in WO2014/064443 or WO2014/064444.

The amphiphilic molecules may be chemically-modified or functionalized to facilitate coupling of the polynucleotide.

The amphiphilic layer may be a monolayer or a bilayer. The amphiphilic layer is typically planar. The amphiphilic layer may be curved. The amphiphilic layer may be supported. The amphiphilic layer may be concave. The amphiphilic layer may be suspended from raised pillars such that the peripheral region of the amphiphilic layer (which is attached to the pillars) is higher than the amphiphilic layer region. This may allow the microparticle to travel, move, slide or roll along the membrane as described above.

Amphiphilic membranes are typically naturally mobile, essentially acting as two-dimensional fluids with lipid diffusion rates of approximately 10⁻⁸ cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in WO 2008/102121, WO 2009/077734 and WO 2006/100484.

Methods for forming lipid bilayers are known in the art. Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed. Planar lipid bilayers may be formed across an aperture in a membrane or across an opening into a recess.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface.

For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent leads to formation of a lipid bilayer. However, complete removal of the solvent from the bilayer is difficult and consequently the bilayer formed by this method is less stable and more prone to noise during electrochemical measurement.

Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface.

Liposomes can be formed by sonication, extrusion or the Mozafari method (Colas et al. (2007) Micron 38:841-847).

In a preferred embodiment, the lipid bilayer is formed as described in WO 2009/077734. Advantageously in this method, the lipid bilayer is formed from dried lipids. In a most preferred embodiment, the lipid bilayer is formed across an opening as described in WO2009/077734.

A lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Any lipid composition that forms a lipid bilayer may be used. The lipid composition is chosen such that a lipid bilayer having the required properties, such as surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally-occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moicties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis-9-Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester. The lipids may be mycolic acid.

The lipids can also be chemically-modified. The head group or the tail group of the lipids may be chemically-modified. Suitable lipids whose head groups have been chemically-modified include, but are not limited to, PEG-modified lipids, such as 1,2-Diacyl-sn-Glycero-3-Phosphoethanolamine-N-[Methoxy(Polyethylene glycol)-2000]; functionalized PEG Lipids, such as 1.2-Distearoyl-sn-Glycero-3 Phosphoethanolamine-N-[Biotinyl(Polyethylene Glycol)2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine. The lipids may be chemically-modified or functionalised to facilitate coupling of the polynucleotide.

The amphiphilic layer, for example the lipid composition, typically comprises one or more additives that will affect the properties of the layer. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides.

Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon or elastomers such as two-component addition-cure silicone rubber, and glasses. The solid state layer may be formed from graphene. Suitable graphene layers are disclosed in WO 2009/035647. Yusko et al., Nature Nanotechnology, 2011; 6: 253-260 and US Patent Application No. 2013/0048499 describe the delivery of proteins to transmembrane pores in solid state layers without the use of microparticles. The method of the invention may be used to improve the delivery in the methods disclosed in these documents.

The method is typically carried out using (i) an artificial amphiphilic layer comprising a pore, (ii) an isolated, naturally-occurring lipid bilayer comprising a pore, or (iii) a cell having a pore inserted therein. The method is typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the invention is typically carried out in vitro.

The membrane to which the polynucleotide is delivered according to the method of the invention is contained in a liquid. The liquid keeps the membrane “wet” and stops it drying out. The liquid is typically an aqueous solution. The aqueous solution typically has the same density as water. The density of the aqueous solution is typically about 1 g/cm³. The density of the solution may vary depending on temperature and the exact composition of the solution. The aqueous solution typically has a density between about 0.97 and about 1.03 g/cm³.

The membrane typically separates two volumes of aqueous solution. The membrane resists the flow of electrical current between the volumes. The transmembrane pore inserted into the membrane selectively allows the passage of ions across the membrane, which can be recorded as an electrical signal detected by electrodes in the two volumes of aqueous solution. The presence of the target polynucleotide modulates the flow of ions and is detected by observing the resultant variations in the electrical signal.

### Polynucleotide Binding Proteins

Aspects of the disclosure relate to methods, compositions and systems comprising one or more polynucleotide binding proteins. A polynucleotide binding protein (e.g., polynucleotide unwinding enzyme) may be any protein that is capable of binding to the polynucleotide and controlling its movement through the pore.

Any of the methods described herein may comprise the step of controlling the movement (for example translocation) of a single-stranded nucleic acid (for example a single-stranded nucleic acid originally comprised in a double-stranded nucleic acid complex as described herein) through a nanopore.

It is straightforward in the art to determine whether or not a protein binds to a polynucleotide. The protein typically interacts with and modifies at least one property of the polynucleotide. The protein may modify the polynucleotide by cleaving it to form individual nucleotides or shorter chains of nucleotides, such as di- or trinucleotides. The moiety may modify the polynucleotide by orienting it or moving it to a specific position, i.e. controlling its movement.

The polynucleotide binding protein (e.g., polynucleotide unwinding enzyme) is preferably derived from a polynucleotide handling enzyme. A polynucleotide handling enzyme is a polypeptide that is capable of interacting with and modifying at least one property of a polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. The polynucleotide handling enzyme does not need to display enzymatic activity as long as it is capable of binding the polynucleotide and controlling its movement through the pore. For instance, the enzyme may be modified to remove its enzymatic activity or may be used under conditions which prevent it from acting as an enzyme. Such conditions are discussed in more detail below.

Typically, the polynucleotide binding protein is a helicase, a polymerase, an exonuclease, a topoisomerase, or a variant thereof.

The polynucleotide handling enzyme is preferably derived from a nucleolytic enzyme. The polynucleotide handling enzyme used in the construct of the enzyme is more preferably derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31. The enzyme may be any of those disclosed in WO 2010/086603.

Preferred enzymes are polymerases, helicases, translocases and topoisomerases, such as gyrases. The polymerase may be PyroPhage 3173 DNA Polymerase (which is commercially available from Lucigen Corporation), SD Polymerase (commercially available from Bioron) or variants thereof. The polymerase is preferably Phi29 DNA polymerase or a variant thereof. The topoisomerase is preferably a member of any of the Moiety Classification (EC) groups 5.99.1.2 and 5.99.1.3.

The enzyme is most preferably derived from a helicase. The helicase may be or be derived from a He1308 helicase, a RecD helicase, such as TraI helicase or a TrwC helicase, a XPD helicase or a Dda helicase. The helicase may be or be derived from He1308 Mbu, He1308 Csy He1308 Tga, He1308 Mhu, TraI Eco, XPD Mbu or a variant thereof.

The helicase may be any of the helicases, modified helicases or helicase constructs disclosed in WO 2013/057495, WO 2013/098562, WO2013098561, WO 2014/013260, WO 2014/013259, WO 2014/013262 and WO/2015/055981.

The Dda helicase preferably comprises any of the modifications disclosed in WO/2015/055981 and WO 2016/055777.

Any number of helicases may be used in accordance with the invention. For instance, 1, 2. 3, 4, 5, 6, 7, 8, 9, 10 or more helicases may be used. In some embodiments, different numbers of helicases may be used. Any combination of two or more of the helicases mentioned above may be used. The two or more helicases may be two or more Dda helicases. The two or more helicases may be one or more Dda helicases and one or more TrwC helicases. The two or more helicases may be different variants of the same helicase.

The two or more helicases are preferably attached to one another. The two or more helicases are more preferably covalently attached to one another. The helicases may be attached in any order and using any method. Preferred helicase constructs for use in the invention are described in WO 2014/013260, WO 2014/013259, WO 2014/013262 and WO2015/055981.

In some embodiments, the polynucleotide binding protein is a polynucleotide unwinding enzyme. A polynucleotide unwinding enzyme is an enzyme that is capable of unwinding a double-stranded polynucleotide into single stranded. In some embodiments, the polynucleotide unwinding enzyme is capable of unwinding a double stranded DNA into single strands. In some embodiments, a polynucleotide unwinding enzyme is an enzyme that possesses helicase activity. Examples of polynucleotide unwinding enzyme include, e.g., helicases described herein.

Polynucleotide binding ability can be measured using any method known in the art. For instance, the protein can be contacted with a polynucleotide and its ability to bind to and move along the polynucleotide can be measured. The protein may include modifications that facilitate binding of the polynucleotide and/or facilitate its activity at high salt concentrations and/or room temperature. Proteins may be modified such that they bind polynucleotides (i.e. retain polynucleotide binding ability) but do not function as a helicase (i.e. do not move along polynucleotides when provided with all the necessary components to facilitate movement, (e.g. ATP and Mg²⁺). Such modifications are known in the art. For instance, modification of the Mg²⁺ binding domain in helicases typically results in variants which do not function as helicases. These types of variants may act as molecular brakes.

The enzyme may be covalently attached to the pore. Any method may be used to covalently attach the enzyme to the pore.

In strand sequencing, the polynucleotide is translocated through the pore either with or against an applied potential. Exonucleases that act progressively or processively on double stranded polynucleotides can be used on the cis side of the pore to feed the remaining single strand through under an applied potential or the trans side under a reverse potential. Likewise, a helicase that unwinds the double stranded DNA can also be used in a similar manner. A polymerase may also be used. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential.

Any helicase may be used in the invention. Helicases may work in two modes with respect to the pore. First, the method is preferably carried out using a helicase such that it moves the polynucleotide through the pore with the field resulting from the applied voltage. In this mode the 5′ end of the polynucleotide is first captured in the pore, and the helicase moves the polynucleotide into the pore such that it is passed through the pore with the field until it finally translocates through to the trans side of the membrane. Alternatively, the method is preferably carried out such that a helicase moves the polynucleotide through the pore against the field resulting from the applied voltage. In this mode the 3′ end of the polynucleotide is first captured in the pore, and the helicase moves the polynucleotide through the pore such that it is pulled out of the pore against the applied field until finally ejected back to the cis side of the membrane.

The method may also be carried out in the opposite direction. The 3′ end of the polynucleotide may be first captured in the pore and the helicase may move the polynucleotide into the pore such that it is passed through the pore with the field until it finally translocates through to the trans side of the membrane.

When the helicase is not provided with the necessary components to facilitate movement or is modified to hinder or prevent its movement, it can bind to the polynucleotide and act as a brake slowing the movement of the polynucleotide when it is pulled into the pore by the applied field. In the inactive mode, it does not matter whether the polynucleotide is captured either 3′ or 5′ down, it is the applied field which pulls the polynucleotide into the pore towards the trans side with the enzyme acting as a brake. When in the inactive mode, the movement control of the polynucleotide by the helicase can be described in a number of ways including ratcheting, sliding and braking. Helicase variants which lack helicase activity can also be used in this way.

The polynucleotide may be contacted with the polynucleotide binding protein (e.g., polynucleotide unwinding enzyme) and the pore in any order. It is preferred that, when the polynucleotide is contacted with the polynucleotide binding protein (e.g., polynucleotide unwinding enzyme), such as a helicase, and the pore, the polynucleotide firstly forms a complex with the polynucleotide binding protein (e.g., polynucleotide unwinding enzyme). When the voltage is applied across the pore, the polynucleotide/polynucleotide binding protein (e.g., polynucleotide unwinding enzyme) complex then forms a complex with the pore and controls the movement of the polynucleotide through the pore.

The polynucleotide binding protein may be modified to prevent the polynucleotide binding protein disengaging from the polynucleotide. Thus, the target polynucleotide preferably does not disengage from the polynucleotide binding protein.

As used herein, the term “disengaging” refers to the dissociation of the polynucleotide binding protein from the target polynucleotide. Thus, a polynucleotide binding protein may be modified to prevent it from dissociating from the target polynucleotide, e.g., into the reaction medium. It is important to distinguish potential “disengagement” of a polynucleotide binding protein from “unbinding” of a polynucleotide binding protein from a target polynucleotide. As used herein, “unbinding” refers to the transient release of the target polynucleotide the active site of the polynucleotide binding protein (described in more detail herein) but does not imply disengagement. Thus, for example, a polynucleotide binding protein may be modified to prevent the polynucleotide binding protein from disengaging from a polynucleotide, but without preventing the polynucleotide binding protein from unbinding from the polynucleotide. When unbound, the polynucleotide binding protein remains engaged with the target polynucleotide. For example, the polynucleotide binding protein may remain engaged with the target polynucleotide (i.e., it may be prevented from disengaging from the target polynucleotide) because it is topologically closed around the target polynucleotide. The polynucleotide binding site may remain free to bind or unbind the target polynucleotide such that the polynucleotide binding protein may bind or unbind to the target polynucleotide, whilst the polynucleotide binding protein remains engaged with the target polynucleotide. When the polynucleotide binding protein is unbound from the target polynucleotide it may be able to move on (e.g., along) the target polynucleotide under an applied force and may be capable of re-binding to the target polynucleotide. When engaged on the target polynucleotide but unbound from the target polynucleotide, the polynucleotide binding protein is not capable of dissociating from the target polynucleotide.

The polynucleotide binding protein can be adapted to prevent disengagement in any suitable way. For example, the polynucleotide binding protein can be loaded on the polynucleotide and then modified in order to prevent it from disengaging from the polynucleotide. Alternatively, the polynucleotide binding protein can be modified to prevent it from disengaging from the polynucleotide before it is loaded onto the polynucleotide. Modification of a polynucleotide binding protein and/or a polynucleotide binding protein in order to prevent it from disengaging from a polynucleotide can be achieved using methods known in the art, such as those discussed in WO 2014/013260, which is hereby incorporated by reference in its entirety, and with particular reference to passages describing the modification of polynucleotide binding proteins such as helicases in order to prevent them from disengaging with polynucleotide strands. For example, a polynucleotide binding protein can be modified by treating with tetramethylazodicarboxamide (TMAD). Various other closing moieties are described in WO 2021/255476 (incorporated herein by reference in its entirety).

For example, a polynucleotide binding protein and/or a polynucleotide binding protein may have a polynucleotide-unbinding opening, e.g., a cavity, cleft or void through which a polynucleotide strand may pass when the polynucleotide binding protein disengages from the strand. The polynucleotide-unbinding opening may be the opening through which a polynucleotide may pass when the polynucleotide binding protein disengages from the polynucleotide. The polynucleotide-unbinding opening for a given polynucleotide binding protein can be determined by reference to its structure, e.g., by reference to its X-ray crystal structure. The X-ray crystal structure may be obtained in the presence and/or the absence of a polynucleotide substrate. The location of a polynucleotide-unbinding opening in a given polynucleotide binding protein may be deduced or confirmed by molecular modelling using standard packages known in the art. The polynucleotide-unbinding opening may be transiently produced by movement of one or more parts e.g., one or more domains of the polynucleotide binding protein.

The polynucleotide binding protein may be modified by closing the polynucleotide-unbinding opening. The polynucleotide-unbinding opening may be closed with a closing moiety. Closing the polynucleotide-unbinding opening may therefore prevent the polynucleotide binding protein from disengaging from the polynucleotide. For example, the polynucleotide binding protein may be modified by covalently closing the polynucleotide-unbinding opening. However, as explained above closing the polynucleotide-unbinding opening does not necessarily prevent the target polynucleotide from unbinding from the polynucleotide binding site of the polynucleotide binding protein. A preferred protein for addressing in this way is a helicase.

The polynucleotide binding protein may be modified with a closing moiety for (i) topologically closing the polynucleotide binding site of the polynucleotide binding protein around the target polynucleotide and (ii) promoting unbinding of the target polynucleotide from the polynucleotide binding site of the polynucleotide binding protein and/or retarding re-binding of the target polynucleotide to the polynucleotide binding site of the polynucleotide binding protein. The polynucleotide binding protein may be modified in any suitable manner to facilitate attachment of such a closing moiety.

A closing moiety may comprise a bifunctional cross-linking moiety. The closing moiety may comprise a bifunctional cross-linker. The bifunctional crosslinker may attach at two points on the polynucleotide binding protein and close the polynucleotide-unbinding opening of the polynucleotide binding protein thereby preventing disengagement of the polynucleotide from the polynucleotide binding protein whilst allowing unbinding of the polynucleotide from the polynucleotide-binding site of the polynucleotide binding protein.

The closing moiety may attach at any suitable positions on the polynucleotide binding protein. For example, the closing moiety may crosslink two amino acid residues of the polynucleotide binding protein. Typically, at least one amino acid crosslinked by the closing moiety is a cysteine or a non-natural amino acid. The cysteine or non-natural amino acid may be introduced into the polynucleotide binding protein by substitution or modification of a naturally occurring amino acid residue of the polynucleotide binding protein. Methods for introducing non-natural amino acids are well known in the art and include for example native chemical ligation with synthetic polypeptide strands comprising such non-natural amino acids. Methods for introducing cysteines into a polynucleotide binding protein are likewise within the capability of one of skill in the art, for example using techniques disclosed in references such as Sambrook et al., Molecular Cloning: A Laboratory Manual, 4th ed., Cold Spring Harbor Press, Plainsview, New York (2012); and Ausubel et al., Current Protocols in Molecular Biology (Supplement 114), John Wiley & Sons, New York (2016).

The closing moiety may have a length of from about 1 Å to about 100 Å. The length of the closing moiety may be calculated according to static bond lengths or more preferably using molecular dynamics simulations. The length may for example be from about 2 Å to about 80 Å, such as from about 5 Å to about 50 Å, e.g., from about 8 to about 30 Å such as from about 10 to about 25 Å or about 20 Å, e.g., about 1, 2, 3, 4, 5, 6, 7, 8, 9, 10, 11, 12, 13, 14, 15, 16, 17, 18, or 19 Å.

Polynucleotide binding proteins suitable for being closed using a closing moiety as described above are discussed in more detail herein. The polynucleotide binding protein is preferably a helicase, e.g., a Dda helicase as described herein.

The polynucleotide binding protein may be or may be derived from an exonuclease. Suitable enzymes include, but are not limited to, exonuclease I from E. coli, exonuclease III enzyme from E. coli, RecJ from T. thermophilus and bacteriophage lambda exonuclease, TatD exonuclease and variants thereof.

The polynucleotide binding protein may be a polymerase. The polymerase may be PyroPhage® 3173 DNA Polymerase (which is commercially available from Lucigen® Corporation), SD Polymerase (commercially available from Bioron®), Klenow from NEB or variants thereof. In one embodiment, the enzyme is Phi29 DNA polymerase or a variant thereof. Modified versions of Phi29 polymerase that may be used in the invention are disclosed in U.S. Pat. No. 5,576,204.

The polynucleotide binding protein may be a topoisomerase. In one embodiment, the topoisomerase is a member of any of the Moiety Classification (EC) groups 5.99.1.2 and 5.99.1.3. The topoisomerase may be a reverse transcriptase, which are enzymes capable of catalysing the formation of cDNA from a RNA template. They are commercially available from, for instance, New England Biolabs® and Invitrogen®.

The polynucleotide binding protein is preferably a helicase. Any suitable helicase can be used in accordance with the method of the invention. For example, the or each enzyme used in accordance with the present disclosure may be independently selected from a Hel308 helicase, a RecD helicase, a TraI helicase, a TrwC helicase, an XPD helicase, and a Dda helicase, or a variant thereof. Monomeric helicases may comprise several domains attached together. For instance, TraI helicases and TraI subgroup helicases may contain two RecD helicase domains, a relaxase domain and a C-terminal domain. The domains typically form a monomeric helicase that is capable of functioning without forming oligomers. Particular examples of suitable helicases include Hel308, NS3, Dda, UvrD, Rep. PcrA, Pifl and TraI. These helicases typically work on single stranded DNA. Examples of helicases that can move along both strands of a double stranded DNA include FtfK and hexameric enzyme complexes, or multisubunit complexes such as RecBCD. The polynucleotide binding protein is preferably a Dda (DNA-dependent ATPase) helicase.

Hel308 helicases are described in publications such as WO 2013/057495, the entire contents of which are incorporated by reference. RecD helicases are described in publications such as WO 2013/098562, the entire contents of which are incorporated by reference. XPD helicases are described in publications such as WO 2013/098561, the entire contents of which are incorporated by reference. Dda helicases are described in publications such as WO 2015/055981 and WO 2016/055777, the entire contents of each of which are incorporated by reference.

The helicase may be Trwc Cba or a variant thereof, Hel308 Mbu or a variant thereof or Dda or a variant thereof. Variants may differ from the native sequences in any of the ways discussed herein. An example variant of Dda comprises E94C/A360C. A further example variant of Dda comprises E94C/A360C and then (ΔM1)G1G2 (i.e., deletion of M1 and then addition of G1 and G2).

### Methods

In some aspects, the disclosure relates to methods of sequencing double stranded nucleic acid complexes by translocating the strands of the complexes through a nanopore and detecting or measuring one or more signals. In some embodiments, the methods comprise measuring a property indicative of the translocation of the first and second nucleic acids of a pair, obtaining data indicative of the measured property, and determining a characteristic of a double stranded nucleic acid complex based upon the obtained data of both the first and second nucleic acids. In some embodiments, the methods comprise detecting a signal corresponding to ion flow through the nanopore to detect polynucleotides of the first and second nucleic acids of a pair translocating through the nanopore; identifying a signal corresponding to translocation of the first nucleic acid of the pair and a sequential signal corresponding to separate translocation of the second nucleic acid of the pair; and analyzing the identified signals, thereby sequencing the double stranded nucleic acid complex.

As used herein, the term “translocate” or “translocation” refers to movement along at least a portion of a nanopore. In some embodiments, translocation is moving from a cis-side of a nanopore to a trans-side of a nanopore.

Ion flow through the transmembrane pore may be monitored using an electrical measurement and/or an optical measurement.

The electrical measurement may be a current measurement, an impedance measurement, a tunneling measurement or a field effect transistor (FET) measurement.

The change in ion flow through the transmembrane pore when the polypeptide translocates through the pore may be detected as a change in current, resistance or an optical property. The effect measured may be electron tunneling across the transmembrane pore. The effect measured may be a change in potential due to the interaction of the polynucleotide with the transmembrane pore wherein the effect is monitored using localized potential sensor in a FET measurement.

A variety of different types of measurements may be made. This includes without limitation: electrical measurements and optical measurements. A suitable optical method involving the measurement of fluorescence is disclosed by J. Am. Chem. Soc. 2009, 131 1652-1653. Possible electrical measurements include current measurements, impedance measurements, tunnelling measurements (Ivanov A P et al., Nano Lett. 2011 Jan. 12; 11(1):279-85), and FET measurements (International Application WO 2005/124888). Optical measurements may be combined with electrical measurements (Soni G V et al., Rev Sci Instrum. 2010 January; 81(1):014301). The measurement may be a transmembrane current measurement such as measurement of ionic current flowing through the pore.

Electrical measurements may be made using standard single channel recording equipment as describe in Stoddart D et al., Proc Natl Acad Sci, 12; 106(19):7702-7, Lieberman K R et al. J Am Chem Soc. 2010; 132(50): 17961-72, and International Application WO 2000/28312. Alternatively, electrical measurements may be made using a multi-channel system, for example as described in WO 2009/077734 and WO 2011/067559.

The method is preferably carried out with a potential applied across the membrane. The applied potential may be a voltage potential. Alternatively, the applied potential may be a chemical potential. In some embodiments, the applied potential may be driven by osmotic imbalance. An example of this is using a salt gradient across a membrane, such as an amphiphilic layer. A salt gradient is disclosed in Holden et al., J Am Chem Soc. 2007 Jul. 11; 129(27):8650-5. In some instances, the current passing through the pore as a polynucleotide moves with respect to the pore is used to estimate or determine the sequence of the polynucleotide.

In some embodiments of various aspects described herein, the method may involve further characterizing the target polynucleotide. As the target polynucleotide is contacted with the pore, one or more measurements which are indicative of one or more characteristics of the target polynucleotide are taken as the polynucleotide moves with respect to the pore.

The method may involve determining whether or not the polynucleotide is modified. The presence or absence of any modification may be measured. The method preferably comprises determining whether or not the polynucleotide is modified by methylation, by oxidation, by damage, with one or more proteins or with one or more labels, tags or spacers. Specific modifications will result in specific interactions with the pore which can be measured using the methods described below. For instance, methylcytosine may be distinguished from cytosine on the basis of the ion flow through the pore during its interaction with each nucleotide.

### Systems

Aspects of the disclosure relate to systems for performing methods described herein. In some embodiments, a system comprises a double-stranded nucleic acid complex each complex comprising a pair of non-covalently bound single stranded nucleic acids, each single stranded nucleic acid of the pair comprising an adaptor having a leader to a nanopore disposed in a membrane, wherein a potential is applied across the membrane to promote translocation of the single stranded nucleic acids through the nanopore, and wherein the system is configured such that the likelihood of nucleic acids of a pair translocating through the nanopore sequentially is greater than the likelihood of nucleic acids from different pairs of non-covalently bound single stranded nucleic acids translocating through the nanopore sequentially. In some embodiments, a system comprises a well (e.g., a well of a sequencing apparatus) comprising a nanopore disposed in a membrane; a plurality of tethers, wherein the concentration of the plurality of tethers added to the well is at least 100 nM; a double stranded nucleic acid molecule comprising a first strand hybridized to a complementary second strand, each strand comprising a leader sequence comprising at least two non-continuous poly-dT sections.

The systems may be part of any apparatus that is suitable for investigating a membrane/pore system in which a pore is present in a membrane. The methods may be carried out using any apparatus that is suitable for transmembrane pore sensing. For example, the apparatus comprises a chamber comprising an aqueous solution and a barrier that separates the chamber into two sections. The barrier typically has an aperture (e.g., a well) in (or across) which the membrane containing the pore is formed. Alternatively the barrier forms the membrane in which the pore is present.

The methods may be carried out using the apparatus described in WO 2008/102120. A variety of different types of measurements may be made. This includes without limitation: electrical measurements and optical measurements. A suitable optical method involving the measurement of fluorescence is disclosed by J. Am. Chem. Soc. 2009, 131 1652-1653. Possible electrical measurements include: current measurements, impedance measurements, tunnelling measurements (Ivanov A P et al., Nano Lett. 2011 Jan. 12; 11(1):279-85), and FET measurements (International Application WO 2005/124888). Optical measurements may be combined with electrical measurements (Soni G V et al., Rev Sci Instrum. 2010 January; 81(1):014301). The measurement may be a transmembrane current measurement such as measurement of ionic current flowing through the pore.

Electrical measurements may be made using standard single channel recording equipment as describe in Stoddart D et al., Proc Natl Acad Sci, 12; 106(19):7702-7, Lieberman K R et al. J Am Chem Soc. 2010; 132(50):17961-72, and International Application WO 2000/28312. Alternatively, electrical measurements may be made using a multi-channel system, for example as described in International Application WO 2009/077734 and International Application WO 2011/067559.

The method is preferably carried out with a potential applied across the membrane. The applied potential may be a voltage potential. Alternatively, the applied potential may be a chemical potential. An example of this is using a salt gradient across a membrane, such as an amphiphilic layer. A salt gradient is disclosed in Holden et al., J Am Chem Soc. 2007 Jul. 11; 129(27):8650-5. In some instances, the current passing through the pore as a polynucleotide moves with respect to the pore is used to estimate or determine the sequence of the polynucleotide.

The methods may involve measuring the current passing through the pore as the polynucleotide moves with respect to the pore. Therefore the apparatus may also comprise an electrical circuit capable of applying a potential and measuring an electrical signal across the membrane and pore. The methods may be carried out using a patch clamp or a voltage clamp. The methods preferably involve the use of a voltage clamp.

The methods of the invention may involve the measuring of a current passing through the pore as the polynucleotide moves with respect to the pore. Suitable conditions for measuring ionic currents through transmembrane protein pores are known in the art and disclosed in the Example. The method is typically carried out with a voltage applied across the membrane and pore. The voltage used is typically from +5 V to −5 V, such as from +4 V to −4 V, +3 V to −3 V or +2 V to −2 V. The voltage used is typically from −600 mV to +600 mV or −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The methods are typically carried out in the presence of any charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The charge carriers may be asymmetric across the membrane. For instance, the type and/or concentration of the charge carriers may be different on each side of the membrane.

The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

The methods are typically carried out in the presence of a buffer. In the exemplary apparatus discussed above, the buffer is present in the aqueous solution in the chamber. Any buffer may be used in the method of the invention. Typically, the buffer is phosphate buffer. Other suitable buffers are HEPES and Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The methods may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature. The methods are optionally carried out at a temperature that supports enzyme function, such as about 37° C.

### Data Analysis and Alignment

As described herein, in some embodiments the methods described herein further comprise performing an alignment step. Nucleic acid sequence alignment may be carried out using any one of various alignment methods known in the art, for example such as disclosed in WO2015140535, or Rang et al., Genome Biol 19, 90 (2018).

A full or partial alignment of candidate pairs may be carried out. A minimum alignment overlap may be specified, namely the minimum number of nucleic acids in a pair that are determined to align. The minimum number of pairs, typically base pairs, may be chosen from a value between 20, 50, 100, 500, 1000 or greater.

To determine nucleotide sequences, various methods may be carried out as known in the art, for example such as disclosed in WO 2015/140535, WO 2013/121224, WO 2020/109773 or WO 2018/203084, all of which are hereby incorporated by reference in their entireties.

### Example 1

### Higher Concentrations of Octyl-Tocopherol Tether Increase Follow-on Rates

Genomic DNA from E. coli was amplified and fragmented. Custom sequencing adapters were ligated to the DNA fragments using Oxford Nanopore Technologies' Ligation Sequencing Kit SQK-LSK109, to form a sequencing library.

Electrical measurements were acquired on a GridION flow cell from Oxford Nanopore Technologies. Flow cells were primed with Flush Buffer, and Flush-Tether—containing the tested tether at indicated concentration—was added to the flow cell immediately before addition of the sequencing library. 10 ng of sequencing library was added to the flow cell.

The follow-on rate was determined as the percentage of strands identified as follow-on events.

TABLE 1

| Tether | Follow-on percentage |
|---|---|
| 50 nM OTT-a | 15% |
| 1 μM OTT-a | 25% |
| 50 nM CHOL | 12% |
| 50 nM OTT-b | 12% |

TABLE 2

| | |
|---|---|
| Octyl-tocopherol tether (OTT- | [Octyl-Tocopherol]-8888-TTGACCGCTCGCCTC-[Cy5] |
| a) | 8 = iSp18 spacer |
| Cholesterol tether (CHOL) | [Chol]-8888-TTGACCGCTCGCCTC |
| | 8 = iSp18 spacer |
| Octyl-tocopherol tether (OTT- | [Octyl-Tocopherol]-33-TTGACCGCTCGCCTC |
| b) | 3 = iSp3 spacer |

The custom sequencing adapter comprised the following top strand, including 5′ leader sequence:

3333//99/CTTATTTTTTTATTTTTTTATTTT/3/CTACATCTCCTTAT
TCGCTGCAC/333/TTmUmUTT/8/CCTGTACTTCGTTCAGTTACGTATT
GCT-N3

where 3=iSpC3, 8=iSp18, mU=2′OMe RNA, 9=iSp9, N3=3′ amino C7 labelled with azidohexanoic acid. Spacers indicated via the codes used by Integrated DNA Technologies, Inc.

### Example 2

### Leaders Comprising One or More Poly-dT Sections Provide Increased Pore Capture

A DNA sequencing library was prepared according to the method described in Example 1. Custom sequencing adapters comprising the following leader sequences were compared:
Poly-T: 33333333TTTTTTTTTTTTTTTTTTTTTTTT
Spacer: 333333333333333333333333333333
3=iSpC3 spacer.

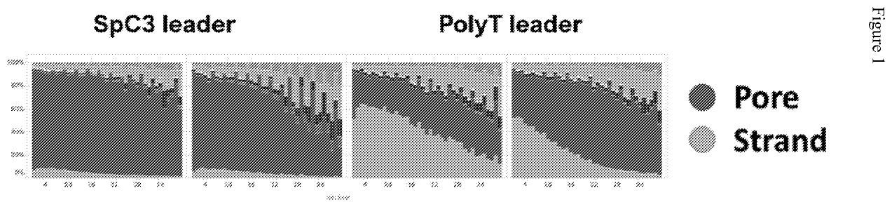

Increased pore capture and resultant sensitivity was observed with the Poly-T leader compared to the Spacer leader (FIG. 1).

### Example 3

### A Longer Hybridisation Length Between Tether and Adaptor Provides Increased Follow-on

A DNA sequencing library was prepared according to the method described in Example 1. Flowcells were primed with 200 nM tether before addition of 3 ng ligated library.

The following tethers were compared:

| | | Length of |
|---|---|---|
| Name | Sequence | hybridisation |
| T1 | [Octyl-Tocopherol]-8- | 25 bp |
| | GTCAGGATTAGTGCGTCTACATGGC | |
| T2 | [Octyl-Tocopherol]-8-GTCAGGATT | 21 bp |
| | AGTGCGTCTACA | |
| T3 | [Octyl-Tocopherol]-8-GTCAGGATT | 16 bp |
| | AGTGCGT | |
| T4 | [Octyl-Tocopherol]-8-GTCAGGATT | 12 bp |
| | AGT | |

8 = iSp18 spacer

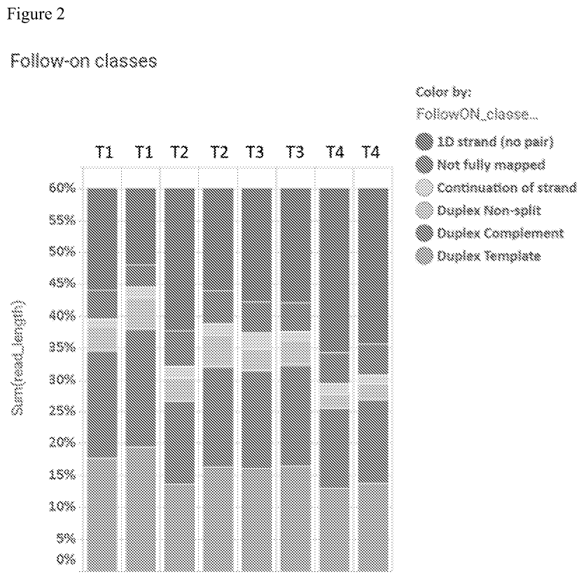

Longer hybridisation sequences gave increased rates of follow-on. The highest rate of follow-on (or “Duplex” data) was achieved with the 25 base pair hybridisation length, which provided approximately 40% follow-on (percentage of all data obtained). Results are presented in FIG. 2.

1. A method comprising:
(i) adding a plurality of tethers to a well comprising a nanopore disposed in a membrane wherein the concentration of tethers added to the well is at least 100 nM;
(ii) contacting the nanopore with a double stranded nucleic acid complex comprising
a pair of non-covalently bound single stranded nucleic acids, each single stranded nucleic acid of the pair comprising an adaptor having a leader; and
(iii) applying a potential to the membrane to promote translocation of the single stranded nucleic acids through the nanopore.

2. The method of claim 1, wherein the first nucleic acid and the second nucleic acid of the pair are each DNA or RNA.

3. The method of claim 1 or 2, wherein the first nucleic acid and second nucleic acid of the pair are complementary to one another.

4. The method of any one of claims 1 to 3, wherein the adaptor of a first single stranded nucleic acid of the pair is positioned on the 5′ end of the first single stranded nucleic acid and/or the adaptor of a second single stranded nucleic acid of the pair is positioned on the 5′ end of the second single stranded nucleic acid.

5. The method of any one of claims 1 to 4, wherein each leader comprises one or more poly-dT section.

6. The method of claim 5, wherein each leader comprises two or more poly-dT sections, optionally wherein each of the poly-dT sections are non-contiguous.

7. The method of any one of claims 1 to 6, wherein each adaptor further comprises one or more spacers.

8. The method of claim 7, wherein each of the one or more spacers are selected from an iSp3C spacer, iSpC9 spacer, and iSpC18 spacer.

9. The method of any one of claims 1 to 8, wherein each adaptor further comprises one or more modified nucleotides, optionally wherein the modified nucleotides are 2′-O-Methyl (2′OMe) modified nucleotides.

10. The method of any one of claims 1 to 9, wherein the nanopore is a protein nanopore, optionally wherein the nanopore is a CsgG nanopore.

11. The method of any one of claims 1 to 10, wherein each of the tethers is a lipid, fatty acid, sterol, carbon nanotube, polypeptide, protein or amino acid.

12. The method of claim 11, wherein each of the tethers comprises tocopherol, optionally wherein each of the tethers comprises octyl-tocopherol.

13. The method of any one of claims 1 to 12, wherein the concentration of tethers added to the well comprises between about 100 nM and 1 μM, 500 nM and 2 μM, 1 μM and 10 μM, or 5 μM and 50 μM.

14. The method of any one of claims 1 to 13 further comprising a step of measuring a property indicative of the translocation of the first and second nucleic acids of the pair, obtaining data indicative of the measured property, and determining a characteristic of the double stranded nucleic acid complex based upon the obtained data of both the first and second nucleic acids.

15. The method of any one of claims 1 to 14 further comprising (iv) detecting a signal corresponding to ion flow through the nanopore to detect polynucleotides of the first and second nucleic acids translocating through the pore; (v) identifying a signal corresponding to translocation of the first nucleic acid of the pair and a sequential signal corresponding to separate translocation of the second nucleic acid of the pair; and (vi) analyzing the signals identified in (v), thereby sequencing the double stranded nucleic acid complex.

16. A system comprising a double-stranded nucleic acid complex each complex comprising a pair of non-covalently bound single stranded nucleic acids, each single stranded nucleic acid of the pair comprising an adaptor having a leader to a nanopore disposed in a membrane, wherein a potential is applied across the membrane to promote translocation of the single stranded nucleic acids through the nanopore, and wherein the system is configured such that the likelihood of nucleic acids of a pair translocating through the nanopore sequentially is greater than the likelihood of nucleic acids from different pairs of non-covalently bound single stranded nucleic acids translocating through the nanopore sequentially.

17. A system comprising a double-stranded nucleic acid complex each complex comprising a pair of non-covalently bound single stranded nucleic acids, each single stranded nucleic acid of the pair comprising an adaptor having a leader to a nanopore disposed in a membrane, wherein a potential is applied across the membrane to promote translocation of the single stranded nucleic acids through the nanopore, and wherein the membrane comprises a plurality of tethers configured and arranged to promote sequential translocation of members of the pairs of non-covalently bound single stranded nucleic acids through the nanopore at a follow-on read frequency of at least 10 percent.

18. The system of claim 16 or claim 17, wherein the first nucleic acid and the second nucleic acid of the pair are each DNA or RNA.

19. The system of any one of claims 16 to 18, wherein the first nucleic acid and second nucleic acid of the pair are complementary to one another.

20. The system of any one of claims 16 to 19, wherein the adaptor of a first single stranded nucleic acid of the pair is positioned on the 5′ end of the first single stranded nucleic acid and/or the adaptor of a second single stranded nucleic acid of the pair is positioned on the 5′ end of the second single stranded nucleic acid.

21. The system of any one of claims 16 to 20, wherein each leader comprises one or more poly-dT section.

22. The system of claim 21, wherein each leader comprises two or more poly-dT sections, optionally wherein each of the poly-dT sections are non-contiguous.

23. The system of any one of claims 16 to 22, wherein each adaptor further comprises one or more spacers.

24. The system of any one of claims 16 to 23, wherein each of the one or more spacers are selected from an iSp3C spacer, iSpC9 spacer, and iSpC18 spacer.

25. The system of any one of claims 16 to 24, wherein each adaptor further comprises one or more modified nucleotides, optionally wherein the modified nucleotides are 2′-O-Methyl (2′OMe) modified nucleotides.

26. The system of any one of claims 16 to 25, wherein the nanopore is a protein nanopore, optionally wherein the nanopore is a CsgG nanopore.

27. The system of any one of claims 16 to 26, wherein each of the tethers is a lipid, fatty acid, sterol, carbon nanotube, polypeptide, protein or amino acid.

28. The system of claim 27, wherein each of the tethers comprises tocopherol, optionally wherein each of the tethers comprises octyl-tocopherol.

29. The system of any one of claims 16 to 28, wherein the likelihood of nucleic acids of a pair translocating through the nanopore sequentially is at least 15%, 20%, 25%, or 30% greater than the likelihood of nucleic acids from different pairs of non-covalently bound single stranded nucleic acids translocating through the nanopore sequentially.

30. A method for sequentially translocating two non-covalently bound molecules through a nanopore, the method comprising:
(i) contacting a double stranded nucleic acid complex comprising a pair of non-covalently bound single stranded nucleic acids, each single stranded nucleic acid of the pair comprising an adaptor having a leader to a nanopore disposed in a membrane comprising a plurality of tethers, said membrane being contained in a well, wherein the concentration of tethers added to the well is at least 1 μM; and
(ii) applying a potential to the membrane, wherein after application of the potential, the first single stranded nucleic acid translocates through the nanopore, and as the first single stranded nucleic acid translocates, reversibly binding the second single stranded nucleic acid to at least one of the tethers that is present on the membrane, and after the first single stranded nucleic acid of the pair has completely translocated through the nanopore the second single stranded nucleic acid of the pair translocates through the nanopore.

31. A method for sequentially translocating two non-covalently bound molecules through a nanopore, the method comprising:
(i) providing a double stranded nucleic acid complex comprising a pair of non-covalently bound single stranded nucleic acids, each single stranded nucleic acid of the pair comprising an adaptor having a leader;
(ii) contacting the double-stranded nucleic acid complex of (i) to a nanopore disposed in a membrane comprising a plurality of tethers, said membrane being contained in a well, wherein the concentration of tethers added to the well is at least 1 μM, under conditions that promote translocation of a first single stranded nucleic acid of the pair through the nanopore;
(iii) reversibly binding the second single stranded nucleic acid to at least one of the tethers that is present on the membrane; and
(iv) translocating the second single stranded nucleic acid of the pair through the nanopore after the first single stranded nucleic acid of the pair has completely translocated through the nanopore.

32. The method of claim 30 or 31, wherein the first nucleic acid and the second nucleic acid of the pair are each DNA or RNA.

33. The method of any one of claims 30 to 32, wherein the first nucleic acid and second nucleic acid of the pair are complementary to one another.

34. The method of any one of claims 30 to 33, wherein each leader comprises one or more poly-dT section.

35. The method of claim 34, wherein each leader comprises two or more poly-dT sections, wherein each of the poly-dT sections are non-contiguous.

36. The method of any one of claims 30 to 35, wherein each adaptor further comprises one or more spacers.

37. The method of claim 36, wherein each of the one or more spacers are selected from an iSp3C spacer, iSpC9 spacer, and iSpC18 spacer.

38. The method of any one of claims 30 to 37, wherein each adaptor further comprises one or more modified nucleotides, optionally wherein the modified nucleotides are 2′-O-Methyl (2′OMe) modified nucleotides.

39. The method of any one of claims 30 to 38, wherein the nanopore is a protein nanopore.

40. The method of any one of claims 30 to 39, wherein the nanopore is a CsgG nanopore.

41. The method of any one of claims 30 to 40, wherein each of the tethers is a lipid, fatty acid, sterol, carbon nanotube, polypeptide, protein or amino acid.

42. The method of claim 41, wherein each of the tethers comprises tocopherol, optionally wherein each of the tethers comprises octyl-tocopherol.

43. The method of any one of claims 30 to 42, wherein the concentration of tethers added to the well comprises between about 1 μM and 5 μM, 2 μM and 20 μM, or 10 μM and 50 μM.

44. The method of any one of claims 31 to 43, wherein the conditions that promote translocation of a first single stranded nucleic acid of the pair the nanopore comprise applying a potential across the membrane.

45. The method of any one of claims 30 to 44, wherein translocating the second single stranded nucleic acid of the pair through the nanopore comprises capture of the leader of the second single stranded nucleic acid by the nanopore.

46. The method of any one of claims 30 to 45, wherein the second single stranded nucleic acid of the pair translocates through the nanopore immediately after the first single stranded nucleic acid of the pair.

47. The method of any one of claims 30 to 45, wherein one or more nucleic acids that are not part of the complex translocate through the nanopore prior to the second single stranded nucleic acid of the pair translocating through the nanopore.

48. The method of any one of claims 30 to 47, wherein the first single stranded nucleic acid and the second single stranded nucleic acid are no longer non-covalently bound after the first single stranded nucleic acid completely translocates through the nanopore.

49. The method of any one of claims 30 to 49 further comprising a step of measuring a property indicative of the translocation of the first and second nucleic acids of the pair, obtaining data indicative of the measured property, and determining a characteristic of the double stranded nucleic acid complex based upon the obtained data of both the first and second nucleic acids.

50. The method of any one of claims 30 to 49 further comprising (a) detecting a signal corresponding to ion flow through the nanopore to detect polynucleotides of the first and second nucleic acids translocating through the pore; (b) identifying a signal corresponding to translocation of the first nucleic acid of the pair and a sequential signal corresponding to separate translocation of the second nucleic acid of the pair; and (c) analyzing the signals identified in (b), thereby sequencing the double stranded nucleic acid complex.

51. A double stranded nucleic acid complex comprising:
(i) a first single stranded nucleic acid comprising a first template nucleic acid section, and a first adaptor, wherein the first adaptor comprises a leader sequence comprising at least two non-continuous poly-dT sections, wherein the first single stranded nucleic acid is non-covalently bound to a second single stranded nucleic acid comprising a second template nucleic acid section that is complementary to the first template nucleic acid section, and a second adaptor, wherein the second adaptor comprises a leader sequence comprising at least two non-continuous poly-dT sections; and
(ii) a tether.

52. The complex of claim 51, wherein the first template nucleic acid section and/or the second template nucleic acid section is DNA or RNA.

53. The complex of claim 51 or 52, wherein each leader comprises three or more non-continuous poly-dT sections.

54. The complex of any one of claims 51 to 53, wherein each adaptor further comprises one or more spacers.

55. The complex of claim 54, wherein each of the one or more spacers are selected from an iSp3C spacer, iSpC9 spacer, and iSpC18 spacer.

56. The complex of any one of claims 51 to 55, wherein each adaptor further comprises one or more modified nucleotides, optionally wherein the modified nucleotides are 2′-O-Methyl (2′OMe) modified nucleotides.

57. The complex of any one of claims 51 to 56, wherein each of the tethers is a lipid, fatty acid, sterol, carbon nanotube, polypeptide, protein or amino acid.

58. The complex of claim 57, wherein each of the tethers comprises tocopherol, optionally wherein each of the tethers comprises octyl-tocopherol.

59. A system for nucleic acid sequencing comprising a well comprising:
(i) a nanopore disposed in a membrane;
(ii) a plurality of tethers, wherein the concentration of the plurality of tethers added to the well is at least 100 nM;
(iii) a double stranded nucleic acid molecule comprising a first strand hybridized to a complementary second strand, each strand comprising a leader sequence comprising at least two non-continuous poly-dT sections.


SEQUENCE LISTING (9 sequences)

SEQ ID NO: 1 - DNA, 15 bp, Artificial Sequence
  Synthetic Sequence
  misc_feature (1)..(1): 5′-[Octyl-Tocopherol]-8888, wherein 8 is iSp18 spacer
  misc_feature (15)..(15): 3′-Cy5
ttgaccgctcgcctc

SEQ ID NO: 2 - DNA, 15 bp, Artificial Sequence
  Synthetic Sequence
  misc_feature (1)..(1): 5′-[Chol]-8888, wherein 8 is iSp18 spacer
ttgaccgctcgcctc

SEQ ID NO: 3 - DNA, 15 bp, Artificial Sequence
  Synthetic Sequence
  misc_feature (1)..(1): 5′-[Octyl-Tocopherol]-33, wherein 3 is iSp3 spacer
ttgaccgctcgcctc

SEQ ID NO: 4 - DNA, 81 bp, Artificial Sequence
  Synthetic Sequence
  misc_feature (1)..(1): 5′-333399, wherein 3 is iSpC3 and 9 is iSp9
  misc_feature (24)..(25): iSpC3
  misc_feature (47)..(48): 333, wherein 3 is iSpC3
  misc_feature (50)..(50): 2′-O-Methyl RNA
  misc_feature (51)..(51): 2′-O-Methyl RNA
  misc_feature (53)..(54): iSp18
  misc_feature (81)..(81): 3′-amino C7 labelled with azidohexanoic acid
cttatttttttatttttttattttctacatctccttattcgctgcacttuuttcctgtacttcgttcagttacgtattgct

SEQ ID NO: 5 - DNA, 24 bp, Artificial Sequence
  Synthetic Sequence
  misc_feature (1)..(1): 5′-33333333, wherein 3 is iSpC3 spacer
tttttttttttttttttttttttt

SEQ ID NO: 6 - DNA, 25 bp, Artificial Sequence
  Synthetic Sequence
  misc_feature (1)..(1): 5′-[Octyl-Tocopherol]-8, wherein 8 is iSp18 spacer
gtcaggattagtgcgtctacatggc

SEQ ID NO: 7 - DNA, 21 bp, Artificial Sequence
  Synthetic Sequence
  misc_feature (1)..(1): 5′-[Octyl-Tocopherol]-8, wherein 8 is iSp18 spacer
gtcaggattagtgcgtctaca

SEQ ID NO: 8 - DNA, 16 bp, Artificial Sequence
  Synthetic Sequence
  misc_feature (1)..(1): 5′-[Octyl-Tocopherol]-8, wherein 8 is iSp18 spacer
gtcaggattagtgcgt

SEQ ID NO: 9 - DNA, 12 bp, Artificial Sequence
  Synthetic Sequence
  misc_feature (1)..(1): 5′-[Octyl-Tocopherol]-8, wherein 8 is iSp18 spacer
gtcaggattagt